(19) United States
(12) Patent Application Publication
(10) Pub. No.: US 2016/0216351 A1
(43) Pub. Date: Jul. 28, 2016

(54) METHOD FOR DETECTING OXIDATION/REDUCTION REACTION IN VIVO

(71) Applicants: Hideo UTSUMI, Fukuoka (JP); Fuminori HYODO, Fukuoka (JP); Shinji ITO, Fukuoka (JP); KYUSHU UNIVERSITY, NATIONAL UNIVERSITY CORPORATION, Fukuoka (JP)

(72) Inventors: Hideo UTSUMI, Fukuoka (JP); Fuminori HYODO, Fukuoka (JP); Shinji Ito, Fukuoka (JP)

(73) Assignee: Kyushu University, National University Corporation, Fukuoka (JP)

(21) Appl. No.: 14/894,454

(22) PCT Filed: May 29, 2014
(86) PCT No.: PCT/JP2014/064341

§ 371 Date: Mar. 18, 2016

Related U.S. Application Data
(60) Provisional application No. 61/828,442, filed on May 29, 2013.

(51) Int. Cl. G01R 33/48; A61B 5/055; G01R 33/60

(52) U.S. Cl. CPC G01R 33/4828; G01R 33/60; A61B 5/055

(57) ABSTRACT

The object of the invention is to provide a method for detecting an oxidation/reduction reaction of a molecule in a lipophilic portion and visualizing the reaction. This is a method for detecting an oxidation/reduction reaction of a molecule that undergoes a free-radical reaction in a lipid environment, the method including: a step in which a magnetic resonance method is applied to a living body or sample to be examined and a proton image of the molecule that undergoes a free-radical reaction in a lipid environment is thereby obtained; and a step in which the proton image is examined for the image intensity of the living body or sample.

### FIELD OF THE INVENTION

The present invention relates to a method for detecting an oxidation/reduction reaction in vivo and more specifically to a method for detecting an oxidation/reduction reaction of a molecule that undergoes a free radical reaction in a lipid environment.

### BACKGROUND OF THE INVENTION

Currently, diagnostic imaging has been used for diagnosing or treating various diseases. This diagnostic imaging identifies lesions of cancer, cerebral infarction, etc. images morphological changes caused by the diseases, allows to read the characteristics of images and thereby is usefully employed for diagnosing and treating the diseases. On the other hand, in many diseases, changes in bodily functions occur as a result of chronic inflammation at a cellular level before any morphological change occurs as a symptom. Particularly, endogenous molecules, which form free radical intermediates, such as ubiquinone and vitamin K play an important role in homeostasis in vivo, and therefore changes frequently occurs in their dynamics and behavior when diseases occur.

By way of example, ubiquinone is one of electron carriers that are present on mitochondrial inner membranes in all cells as well as on cell membranes of prokaryotes and is deeply involved in maintaining mitochondrial functions. Therefore, ubiquinone is expected to improve mitochondrial functions in cells and show antioxidant effects as well as anti-aldosterone effects and has been used as an adjunctive agent for cardiac functions. Ubiquinone is a molecule involved in accepting and donating electrons, which is called the Q cycle, in mitochondrial respiratory chains I-III, intermediates between respiratory chain complexes I and II for electrons in the electron transfer system and produces semiquinone free radicals in its metabolic process. Such free radicals are related to a biological redox reaction. The biological redox reaction is a concept representing all of the expression of physiological functions via oxidation/reduction reactions and the production of activated species associated therewith as well as the metabolism and reaction of activated species thus produced and biological molecules, and it is suggested that the biological redox reaction is deeply involved in many physiological phenomena as well as biological redox diseases such as cancer and diabetics.

Accordingly, it is believed that if there is a method for directly visualizing the behavior and state of an oxidation/reduction reaction of an endogenous molecule such as ubiquinone, it becomes possible to diagnose and treat various diseases and elucidate the mechanism of those diseases on the basis of information about such an endogenous molecule.

Conventional methods of biological imaging include X-ray CT, CT and magnetic resonance imaging (MRI), i.e., morphological imaging in which spatial information is imaged has mainly been performed conventionally. In addition to morphological imaging, functional imaging in which biological functions and phenomena are visualized by PET, etc. has recently been performed.

For example, there is a case in which a free radical produced in a solution prepared from an extracted organ was measured by an electron spin resonance method or the like and its function was analyzed on the basis of the waveform of its spectrum and changes in intensity. This method could not elucidate as to when, where and how a biological substance is involved in a disease, though analyses could be made at a test tube level.

As a method for detecting and analyzing an oxidation/reduction reaction in vivo, it has been known that a synthetic nitroxyl radical compound is administered to the living body as a probe (contrast agent) and detection and analyses are made by using an oxidation/reduction reaction of the compound as a reference. However, this method only detects the disappearance of nitroxyl radicals and, therefore, is simply detecting and analyzing an oxidation/reduction reaction in vivo on the basis of the reaction of the synthetic nitroxyl radical compound as a reference. Therefore, this method is not to directly detect and analyze an oxidation/reduction reaction of an endogenous molecule. Moreover, it is difficult to obtain sufficient image intensity of nitroxyl radicals in an organic solvent by an image resonance method such as OMRI.

The present inventors have been successful in visualizing an endogenous molecule in a water-soluble environment by an image resonance method (Patent Literature 1). However, they could not succeed in efficient visualization in a fat-soluble environment.

### PRIOR ART LITERATURE

### Patent Literature

Patent Literature 1: International Publication No. 2011/052760

### Non-Patent Literature

Non-Patent Literature 1: Non-invasive monitoring of redox status in mice with dextran sodium sulphate-induced colitis. Yasukawa K, Miyakawa R, Yao T, Tsuneyoshi M, Utsumi H. Free Radic Res. 2009 May; 43 (5):505-13.
Non-Patent Literature 2: In vivo detection of free radicals induced by diethylnitrosamine in rat liver tissue. Yamada K, Yamamiya I. Utsumi H. Free Radic Biol Med. 2006 Jun. 1; 40 (11):2040-6.
Non-Patent Literature 3: Application of in vivo ESR spectroscopy to measurement of cerebrovascular ROS generation in stroke, Yamato M, Egashira T, Utsumi H. Free Radic Biol Med. 2003 Dec. 15; 35 (12):1619-31.

### SUMMARY OF THE INVENTION

The present invention was designed in view of the abovementioned circumstances, and the purpose of the present invention is to provide a method for detecting and visualizing an oxidation/reduction reaction of a molecule in a lipophilic portion in order to discover the initial symptoms of various diseases earlier and make the prevention and treatment of those diseases possible.

The present inventors found that it was possible to obtain image intensity high enough to be detected by a magnetic resonance device even in an organic solvent by using a lipophilic molecule so that its radical body could be used as a contrast agent.

The present inventors conducted extensive study in order to solve the abovementioned problems and, as a result, found that it was possible to detect and visualize an oxidation/reduction reaction of a molecule in a lipid environment by using a magnetic resonance method (including Overhauser MRI and an electron spin resonance method).

Specifically, as the first major viewpoint of the present invention, the invention provides a method for detecting an oxidation/reduction reaction of a molecule that undergoes a free-radical reaction in a lipid environment, the method comprising: a step of applying a magnetic resonance method to a living body or sample to be examined and thereby obtaining a proton image of the molecule that undergoes a free-radical reaction in a lipid environment; and a step of examining the image intensity of the living body or sample in the proton image.

In such a constitution, it is possible to detect an oxidation/reduction reaction of a molecule that undergoes a free-radical reaction in a lipid environment, and therefore the biological functions of living animals can be visualized at lipid sites.

Furthermore, the present invention enables biological functions to be visualized by using a molecule that undergoes a free-radical reaction in a lipid environment, and therefore the preliminary stage of morphological changes caused by diseases can be visualized, which in turn contributes to very early diagnosis and the development of preventive drugs.

Furthermore, according to one embodiment of the present invention, in the abovementioned method, the abovementioned step of obtaining a proton image is to obtain two or more proton images over time, and preferably this method further comprises a step of comparing sequential changes in the image intensity of the abovementioned living body or sample in the abovementioned proton images.

Furthermore, according to one embodiment of the present invention, in the abovementioned method, the abovementioned magnetic resonance method is Overhauser MRI, and the abovementioned step of obtaining a proton image is to obtain a proton image in which an electro spin of the abovementioned molecule that undergoes a free-radical reaction in a lipid environment is excited.

In this case, it is preferred that this method further comprise a step of obtaining a proton image in which an electron spin of the abovementioned molecule that undergoes a free-radical reaction in a lipid environment is not excited; and a step of comparing between a proton image in which an electron spin of the abovementioned molecule that undergoes a free-radical reaction in a lipid environment is excited and a proton image in which an electron spin of the abovementioned molecule that undergoes a free-radical reaction in a lipid environment is not excited and then calculating a difference or percentage of the image intensity of the abovementioned living body or sample in those two images.

Furthermore, according to one embodiment of the present invention, in the abovementioned method, the abovementioned molecule that undergoes a free-radical reaction in a lipid environment is a molecule having a quinone skeleton.

In this case, it is preferred that the abovementioned molecule having a quinone skeleton be selected from the group consisting of ubiquinone (CoQ₁₀), riboflavin, vitamin K₁, vitamin K₂, vitamin K₃, 1,4-benzoquinone (p-quinone), 2,6-dichloro-p-quinone, 1,4-naphthoquinone and seratrodast.

Furthermore, according to one embodiment of the present invention, in the abovementioned method, the abovementioned step of obtaining a proton image is to obtain proton images of two or more molecules that undergo a radical reaction in a lipid environment.

Furthermore, according to one embodiment of the present invention, the abovementioned method further comprises a step of obtaining a proton image of a molecule that undergoes a radical reaction in an aquatic environment.

Furthermore, according to one embodiment of the present invention, in the abovementioned method, the abovementioned living body or sample is administered with a redox material in advance.

In this case, it is preferred that the living body or sample be administered with the abovementioned molecule that undergoes a free-radical reaction in a lipid environment in advance.

Furthermore, according to one embodiment of the present invention, the abovementioned redox material is selected from the group consisting of NaOH, NADH, KO₂ and combinations thereof.

Furthermore, according to one embodiment of the present invention, in the abovementioned method, the abovementioned molecule that undergoes a free-radical reaction in a lipid environment is dissolved in a solvent selected from the group consisting of ethanol, methanol, DMSO, acetone, hexane, chloroform, alkaline solutions and combinations thereof.

The characteristic and remarkable operation and effects of the present invention other than those described above become clear to those skilled in the art by referring to the detailed description of the invention and drawings below.

### BRIEF DESCRIPTION OF THE DRAWINGS

FIG. 1 is images and graphs showing the visualization of an oxidation/reduction reaction by ReMI according to one embodiment of the present invention.

FIG. 2 is images and graphs showing the visualization of an oxidation/reduction reaction by ReMI according to one embodiment of the present invention.

FIG. 3 is images and graphs showing the visualization of an oxidation/reduction reaction in mitochondria by ReMI according to one embodiment of the present invention.

FIG. 4 is images and a graph showing the visualization of an oxidation/reduction reaction in mice by ReMI according to one embodiment of the present invention.

FIG. 5A is an image showing the visualization of a vitamin K₁ free radical by ReMI according to one embodiment of the present invention.

FIG. 5B is a graph showing an X-band ESR spectrum of a vitamin K₁ free radical and a graph showing image intensities according to one embodiment of the present invention.

FIG. 6A is an image showing the visualization of a vitamin K₂ free radical by ReMI according to one embodiment of the present invention.

FIG. 6B is a graph showing an X-band ESR spectrum of a vitamin K₂ free radical and a graph showing image intensities according to one embodiment of the present invention.

FIG. 7A is an image showing the visualization of a vitamin K₃ free radical by ReMI according to one embodiment of the present invention.

FIG. 7B is a graph showing an X-band ESR spectrum of a vitamin K₃ free radical and a graph showing image intensities according to one embodiment of the present invention.

FIG. 8A is an image showing the visualization of a vitamin K₃ free radical by ReMI according to one embodiment of the present invention.

FIG. 8B is a graph showing the image intensity of a vitamin K₃ free radical according to one embodiment of the present invention.

FIG. 9A is an image showing the visualization of vitamin K₂ and vitamin K₃ free radicals by ReMI according to one embodiment of the present invention.

FIG. 9B is a graph showing the image intensity of vitamin K₂ and vitamin K₃ free radicals according to one embodiment of the present invention.

FIG. 10A is an image showing the visualization of a riboflavin (vitamin B₂) free radical by ReMI according to one embodiment of the present invention.

FIG. 10B is a graph showing an X-band ESR spectrum of a riboflavin (vitamin B₂) free radical and a graph showing image intensities according to one embodiment of the present invention.

FIG. 11 is images showing the visualization of an EGCG free radical by ReMI according to one embodiment of the present invention.

FIG. 12 is an image showing the visualization of a dopamine free radical by ReMI according to one embodiment of the present invention.

FIG. 13 is images showing the visualization of a chlorogenic acid free radical by ReMI according to one embodiment of the present invention.

FIG. 14 is images showing the visualization of a caffeic acid free radical by ReMI according to one embodiment of the present invention.

FIG. 15 is images showing the visualization of a rosmarinic acid free radical by ReMI according to one embodiment of the present invention.

FIG. 16 is images showing the visualization of a rutin free radical by ReMI according to one embodiment of the present invention.

FIG. 17 is an image showing the visualization of a seratrodast free radical by ReMI according to one embodiment of the present invention.

FIG. 18 is an image showing the visualization of a trolox free radical by ReMI according to one embodiment of the present invention.

FIG. 19A is an image showing the visualization of TEMPOL by ReMI according to one embodiment of the present invention.

FIG. 19B is a graph showing the image intensity of TEMPOL according to one embodiment of the present invention.

FIG. 20A is images showing the visualization of TEMPOL by ReMI according to one embodiment of the present invention.

FIG. 20B is graphs showing the image intensity of TEMPOL according to one embodiment of the present invention.

FIG. 21A is an image showing the visualization of MC-PROXYL by ReMI according to one embodiment of the present invention.

FIG. 21B a graph showing the image intensity of MC-PROXYL according to one embodiment of the present invention.

FIG. 22A is images showing the visualization of MC-PROXYL by ReMI according to one embodiment of the present invention.

FIG. 22B a graph showing the image intensity of MC-PROXYL according to one embodiment of the present invention.

### DETAILED DESCRIPTION OF THE INVENTION

A description of one embodiment and working examples of the present invention is given below with reference to drawings. In one embodiment of the present invention, the method of the invention is to detect an oxidation/reduction reaction associated with a free-radical reaction in a lipid environment. Here, the “lipid environment” refer to the environment other than an aquatic environment and includes membrane lipid bilayers and lipoproteins, wherein organic solvents are main constituents.

As used herein, the “free-radical reaction” refers to electron transfer in specific atoms, molecules and the like having an unpaired electron. A free-radical has an unpaired electron and is paramagnetic and is involved in a biological redox reaction. The biological redox reaction is a concept representing all of the expression of physiological functions via oxidation/reduction reactions and the production of activated species associated therewith as well as the metabolism and reaction of activated species thus produced and biological molecules, and it is suggested that the biological redox reaction is deeply involved in many physiological phenomena as well as biological redox diseases such as cancer and diabetics. Accordingly, the visualization of the biological redox state can provide a new methodology for analyzing the mechanisms of diseases minimally invasively and developing therapeutic drugs.

As used herein, the “molecule that undergoes a free-radical reaction in a lipid environment” is a molecule that forms a free-radical intermediate in a lipid environment and includes molecules that are present in the living body as well as synthetic compounds. In the case of molecules that are present in the living body, those molecules play an important role in homeostasis in a lipid environment in vivo. The “molecule that undergoes a free-radical reaction in a lipid environment” includes, but is not limited to, CoQ₁₀, riboflavin (vitamin B₂), vitamin K₁ (phylloquinone, 2-methyl-3-phytyl-1,4-naphthoquinone), vitamin K₂ (menaquinone-4, menaquinone-7), vitamin K₃ (menadione, 2-methyl-1,4-naphthoquinone), 1,4-benzoquinone (p-quinone), 2,6-dichloro-p-quinone, 1,4-naphthoquinone, vitamin E (tocopherol (α, β, γ, σ) and tocotrienol (α, β, γ, σ)), trolox, epigallocatechin gallate (EGCG), dopamine, chlorogenic acid, caffeic acid, rosmarinic acid, rutin and seratrodast. Ubiquinone (CoQ₁₀), riboflavin, vitamin K₁, vitamin K₂, vitamin K₃, 1,4-benzoquinone (p-quinone), 2,6-dichloro-p-quinone, 1,4-naphthoquinone and seratrodast are molecules having a quinone skeleton.

By way of example, vitamin K₁ (phylloquinone) forms a free-radical intermediate in the following scheme.

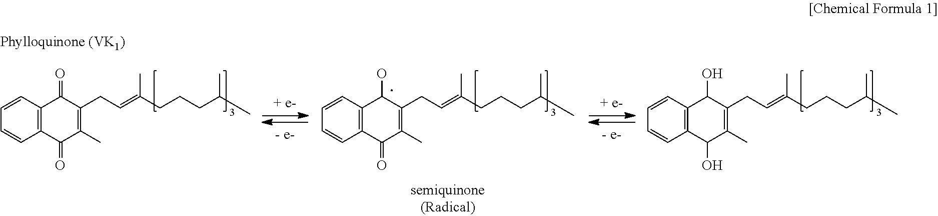

Thus, the “molecule that undergoes a free-radical reaction in a lipid environment” includes molecules that form free-radical intermediates in a lipid environment in vivo such as in a specific cell.

The magnetic resonance method used in the present invention is a generally-used magnetic resonance method in which an phenomenon that when an electromagnetic wave or oscillating field is applied to an object to be examined, a kind of resonance occurs at a specific frequency and the electromagnetic wave is strongly absorbed (magnetic resonance) is used to examine the state of an electron, an atomic nucleus or the like within a material on the basis of the frequency that causes absorption by resonance, the waveform of the absorption or the like. Such magnetic resonance method is exemplified by a magnetic resonance imaging (MRI) method, an Overhauser MRI (OMRI) method, a nuclear magnetic resonance (NMR) method and an electron spin resonance (EPR) method. The measurement conditions of each of the abovementioned magnetic resonance methods can appropriately be selected within the range of conditions generally used in each measuring method. The term “ReMI (Redox Molecular Imaging)” is used herein, and the term has same meaning as OMRI.

As an imaging device using such a magnetic resonance method, a device disclosed in International Publication No. WO 2010/110384, i.e., “a device comprising magnetic field generating means for generating a field for exciting the magnetic resonance of an object to be examined, moving means for moving the object to be examined in the magnetic field of the magnetic field generating means by moving the object to be examined or the magnetic field generating means, measuring means for obtaining a measured image signal within the object to be examined by phase encoding and/or frequency encoding by applying a gradient magnetic field to the moving direction y of the object to be measured relative to the magnetic field generating means and/or the direction x orthogonal to the moving direction y, without stopping during moving by the moving means, and correcting means for obtaining a corrected image signal by correcting the influence of moving in the y direction for the measured image signal” may be used, for example.

For example, at the time of implementing the method of the present invention using ReMI or OMRI, each image can be obtained by turning electron spin irradiation (EPR irradiation, ESR irradiation) on and off. More specifically, electron spin excitation is performed by EPR irradiation for an interested “molecule that undergoes a free-radical reaction in a lipid environment.” As a result, the energy of an electron spin is transferred to a nuclear spin so that the image intensity of a proton increases. By way of example, a peak frequency of the spectrum of a specific radical body is set and then electron spin excitation is performed to obtain an MRI image so that a proton image having increased image intensity can be produced. In the case of no electron spin excitation, a proton image that is produced by ordinary MRI is obtained. As used herein, “EPR (Electron Paramagnetic Resonance)” is synonymous with “ESR (Electron Spin Resonance),” and both indicate electron spin resonance.

In one embodiment of the present invention, when an interested “molecule that undergoes a free-radical reaction in a lipid environment” has not been subjected to an oxidation/reduction reaction yet, image intensity remains high because radicals have not disappeared yet. When radicals disappear as an oxidation/reduction reaction progresses, image intensity declines. Accordingly, the presence or absence of an oxidation/reduction reaction in vivo can be detected by paying attention to a specific “molecule that undergoes a free-radical reaction in a lipid environment” and observing a change in image intensity over time.

Furthermore, in one embodiment of the present invention, while an oxidation/reduction reaction can be detected by using an image taken when electron spin excitation is ON, the oxidation/reduction reaction can also be detected by using two images taken when electron spin excitation is ON and OFF. By way of example, the image intensity of an image taken when EPR irradiation is OFF can be subtracted from the image intensity of an image taken when EPR irradiation is ON (subtraction). An oxidation/reduction reaction can be detected by using the image intensity thus obtained. It is also possible to divide the image intensity of an image taken when EPR irradiation is ON by the image intensity of an image taken when EPR irradiation is OFF (division). An oxidation/reduction reaction may also be detected by using the image intensity thus obtained. Thus, the difference can be emphasized by subtracting or dividing image intensity even when comparison is difficult (e.g., a case in which image intensity is low only based on an image taken when EPR irradiation is ON).

Moreover, in the case of implementing the method of the present invention using MRI, image intensity can also be obtained from information about the relaxation time of water (longitudinal relaxation and traverse relaxation). In MRI, the relaxation time (longitudinal relaxation time: T1 relaxation) is shortened because a radical of a molecule used as a contrast agent interacts with water. Accordingly, in the case of obtaining an image by a T1 weighed imaging method of MRI, image intensity increases for the number of radicals contained in the contrast agent. Hence, image intensity declines when radicals disappear as an oxidation/reduction reaction progresses. In the case of implementing the method of the present invention using MRI, the detection of an oxidation/reduction reaction may be expressed by the percentage of image intensity increased by radicals.

As used herein, the “redox material” functions as an electron donor or an electron acceptor and reacts with the abovementioned molecule that undergoes a free-radical reaction in a lipid environment for an oxidation/reduction reaction. The redox material includes, but is not limited to, NaOH, NADH and KO₂.

Furthermore, in one embodiment of the present invention, the abovementioned molecule that undergoes a free-radical reaction in a lipid environment may be dissolved in an organic medium or an organic solvent. The organic medium or solvent includes, but is not limited to, ethanol, methanol, DMSO, acetone, hexane, chloroform, alkaline solutions and combinations thereof.

Furthermore, in one embodiment of the present invention, in regards to a proton image of a molecule that undergoes a free-radical reaction in a lipid environment, proton images of multiple kinds of molecules that undergo a free-radical reaction in a lipid environment may be obtained at the same time. Proton images of multiple kinds of molecules can be obtained at the same time by adjusting the frequency of EPR irradiation to a region that is in common among multiple kinds of free-radical intermediates. Of course, proton images of multiple kinds of molecules can also be obtained at the same time by continually performing EPR irradiation at a plurality of frequencies and acquiring images on the same specimen. Moreover, the present invention enables to detect an oxidation/reduction reaction of a molecule that undergoes a free-radical reaction in a lipid environment and an oxidation/reduction reaction of a molecule that undergoes a free-radical reaction in an aquatic environment at the same time. In this case, the aquatic environment is a solvent such as water and PBS, wherein a molecule forming a radical body is dissolved in such a solvent. In the case of detecting an oxidation/reduction reaction of a molecule that undergoes a free-radical reaction in a lipid environment and an oxidation/reduction reaction of a molecule that undergoes a free-radical reaction in an aquatic environment at the same time, it is possible to detect electron transfer between the molecule that undergoes a free-radical reaction in a lipid environment and the molecule that undergoes a free-radical reaction in an aquatic environment.

### EXAMPLES

A description of the present invention is given below in more detail with reference to working examples, but the present invention is not limited to those working examples.

### Experimental Approach and Materials

### Free-Radical Intermediates, Phantom and EPR Measurement

Water-soluble intermediates FMNH and FADH were each dissolved in water and prepared by mixing FMN (10 mM) and FAD (18 mM) with the same amount of NADH, respectively. Fat-soluble intermediates CoQ₁₀H, vitamin E and vitamin K₁ radicals were prepared from CoQ₁₀ (10 mM)/acetone/NaOH, vitamin E (1.5M)/hexane/KO₂, and vitamin K₁ (83 mM)/chloroform/ethanol/KO₂, respectively. The EPR spectrum of each free-radical and EPR parameters therefor were obtained by an X-band EPR spectrometer (JEOL Ltd.) at room temperature under the following conditions.

Microwave frequency, 9.4 GHz; microwave power, 1 mW; width modulation, 0.06 mT; sweeping time, 1 minute; sweeping width, +/−5 mT; time constant, 0.03 s.

EPR parameters were calibrated by using the internal standard of Mn²⁺. In the ReMI experiment, the apparent concentration of each free-radical intermediate was found by extrapolating a time-dependent curve of an EPR spectral region on the basis of a CmP peak region and the internal standard of Mn²⁺.

### Instrument for ReMI

The ReMI experiment was conducted by using a DNP-MRI system manufactured at Kyushu University. The DNP-MRI system was constituted by using an external magnet for an EPR device (JES-ES20, JEOL Ltd.) and two axis field gradient coils for CW-EPR imaging. A resonator was constituted of a surface coil for ESR irradiation, an NMR cross coil within a saddle, and a solenoid for transmission and signal reception. An ESR irradiation coil was disposed between two NMR coils. The external magnetic field B₀ for EPR irradiation and MRI was fixed at 20 mT, and high frequency waves for EPR irradiation and MRI were 527.5 MHz and 793 kHz, respectively. A surface coil (diameter: 20 mm) was used for ESR irradiation, and an NMR coil assembly was constituted of an NMR transmission saddle coil (90 mm i.d., 175 mm in length) and a solenoid reception coil (40 mm i.d., 60 mm in length) having a bandwidth of 1 kHz. The maximum transmission power was 100 W. The ReMI experiment was conducted by a spin echo method. The ReMI experiment was conducted under the following conditions: EPR irradiation power, 12 W; flip angle, 90 degrees; T_{EPR}×repetition time (T_{R})×echo time (T_{E})=500×1000×40 ms; average number=1; slice thickness, 30 mm; and a 64-phase modulation step. For the image field (32×32 mm), 64×64 matrix was used.

### Spectroscopic Imaging of Free-Radical Intermediates Using ReMI

The phantom is made of four tubes containing CoQ₁₀H, FMNH, ¹⁴N and ¹⁵N-labelled CmPs. CoQ₁₀H and FMNH were prepared as described above. In the ReMI experiment, EPR irradiation was performed using the abovementioned ReMI system at a specific frequency between 500 MHz and 580 MHz.

### Metabolic Imaging in the Presence of Mitochondria

To a phantom tube filled with mitochondria collected from a rat was added FADH or CoQ₀H in an experiment. One sample was inactivated by heating. In the ReMI experiment, EPR irradiation was performed at 572.5 MHz and the ReMI system was used as described above, and images were examined every two minutes after starting the reaction with mitochondria. The ReMI experiment was conducted under the following conditions: EPR irradiation, 12 W; flip angle, 90 degrees; T_{EPR}×repetition time (T_{R})×echo time (T_{E})=500×1000×40 ms; average number=1; slice thickness, 30 mm; 64-phase encoding; scanning time, 70 seconds. The metabolic rates (decreasing rates) of FADH and CoQ₀H were calculated from changes in the first four image intensities after the reaction with mitochondria.

### Metabolic Imaging in Mice

Female C57BL6 mice (5 weeks old) were purchased from Japan SLC, Inc. (Hamamatsu, Japan) and acclimated for one week before the experiment. Mice were 6 to 8 weeks old at the time of the experiment (body weight: 20-30 g), and five mice were kept in each cage in a room that was adjusted to a 24-hour cycle rhythm, wherein the temperature and moisture of the room were also adjusted. Food and water were given ad libitum. All the procedures and animal care were approved by the Animal Experiment Ethics Committee of Kyushu University and carried out in accordance with guidelines for animal experiments established by Kyushu University.

Mice were anesthetized with 2% isoflurane in the FADH experiment or with urethane (2 g/kg) in the CoQ₀H experiment and immobilized with a skin adhesive tape, wherein the stomach was placed to the lower side. During the experiment, the temperature of mice was kept at 37+/−1° C. with a warm current of air. Subsequently, mice were placed in a resonator and ReMI measurement was started. ReMI images of the lower abdominal region were examined after administering an 8 mM CoQ₀ alkaline solution (800 μL) to the rectum or administering an FAD/NADH solution intramuscularly.

ReMI images were obtained by using a DNP-MRI system manufactured at Kyushu University in an experiment using FADH and a Philips prototype system in an experiment using CoQ₀. The ReMI experiment was conducted using the abovementioned parameters. Radical metabolic images (redox maps) were obtained by calculating changes in ReMI intensity in each pixel among the first four ReMI images (from a semi-logarithmic plot line of each pixel on sequential images).

### Image Analysis

ReMI data was analyzed by using Image J software (http://rsb.info.nih.gov/ij/).

### Experiment Results

A description of experiment results is given below with reference to drawings.

### 1. Simultaneous Visualization of Endogenous Molecules by ReMI

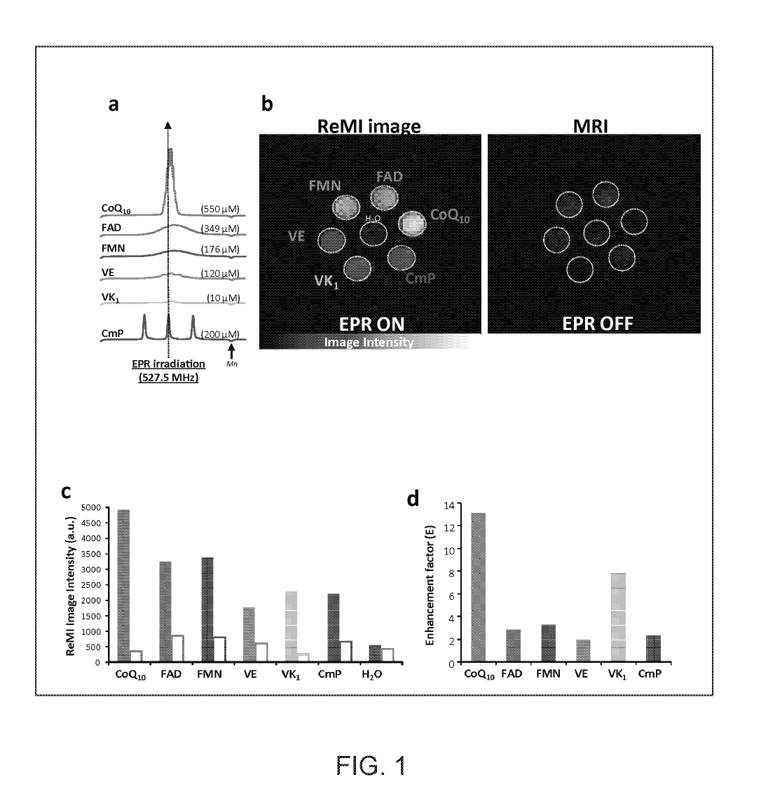

Seven phantoms containing free-radicals derived from FMNH, FADH, CoQ₁₀H, vitamin E and vitamin K₁ and a synthetic CmP free-radical were designed. FMNH, FADH and CmP were dissolved in a water-soluble solvent, and CoQ₁₀H, vitamin E and vitamin K₁ free-radicals were dissolved in a fat-soluble solvent. Item a in FIG. 1 shows each EPR spectrum. The concentration of each free-radical (as shown on the right side of each spectrum in item a in FIG. 1) was determined by X-band ESR (27-550 μM). Ordinary MRI images had low image intensity (right in item b in FIG. 1). EPR irradiation in the ReMI experiment was conducted at 527.5 MHz (as shown by a perpendicular line in item a in FIG. 1) with 10 W continuous waves, wherein the perpendicular line shows the central peak of the synthetic CmP. In ReMI images, all of the endogenous free-radical intermediates and synthetic CmP showed different image intensities (left in item b in FIG. 1). The ReMI image of each free-radical intermediate is derived from solvent protons (FMNH, FADH and CmP were from water protons and CoQ₁₀H, vitamin E and vitamin K₁ free-radicals from hydrocarbon protons). Although they were more complicated and had wider lines (FIG. 1) than the EPR spectrum of CmP, the EPR spectra of the free-radical intermediates of the endogenous compounds could be imaged by ReMI. Item c in FIG. 1 shows the image intensities of those phantoms with DNP and no DNP, and item d in FIG. 1 shows their enhancement factors (intensity ratios based on EPR irradiation/no irradiation).

### 2. Spectroscopic 2D Imaging of Free-Radical Intermediates in Single ReMI Experiment

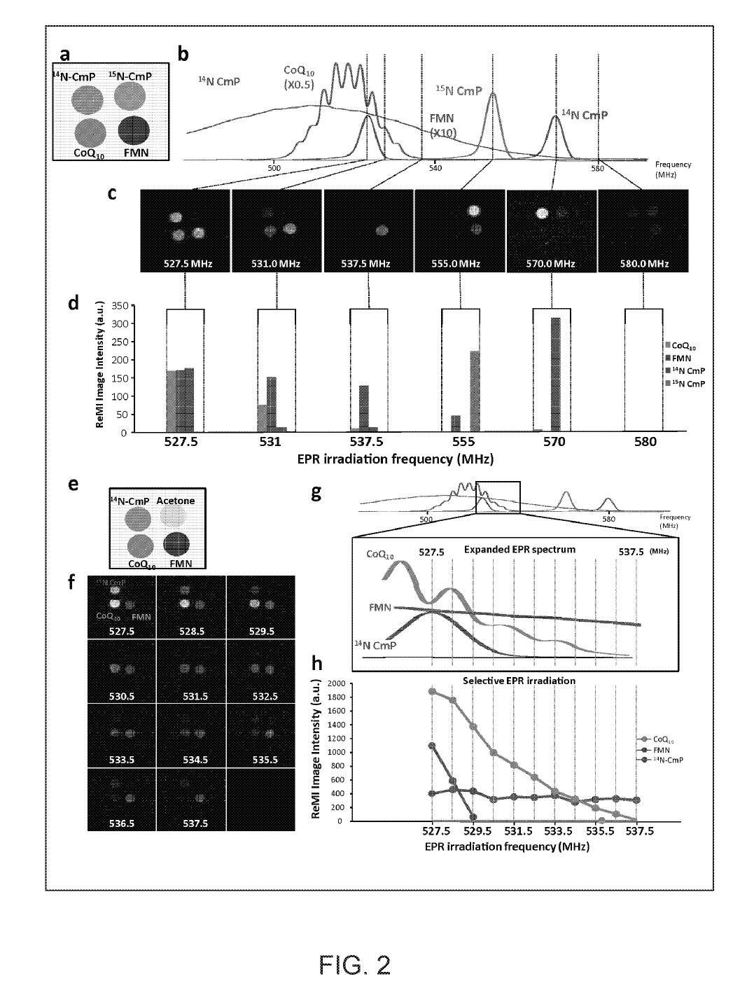

The present inventors reported that ReMI enabled to perform imaging of multiple species, just like the chemical shift in MRI or magnetic resonance spectroscopic imaging (MRSI). While images in FIG. 1 were obtained by irradiating a single frequency for all of the free-radical species, the present inventors tested the ReMI capability for distinguishing several free-radical species in a given field by using phantoms containing CoQ₁₀H, FMNH and synthetic ¹⁴N- or ¹⁵N-labelled CmP (item a in FIG. 2). In item b in FIG. 2, individual EPR absorption spectra of those species were superimposed along the spectral range of 500-580 MHz. Image data were obtained by using pulse sequences described in the abovementioned methodology (item c in FIG. 2). By changing the frequency of EPR irradiation, discrete images of free-radical intermediates FMNH, CoQ₁₀H and ¹⁴N-CmP could be visualized in the ReMI experiment. For CoQ₁₀M and ¹⁴N-CmP, clear images were obtained at 527.5 MHz and images were unclear at 531 MHz. On the other hand, signals of FMNH were clear at 527.5-537.5 MHz. Just like the previous observation made by the present inventors, ¹⁵N- and ¹⁴N-labelled free-radicals could be visualized by using EPR irradiation at 555 MHz and 570 MHz, respectively. Item d in FIG. 2 shows the intensities of paramagnetic intermediates at each EPR irradiation frequency. This result shows that each free-radical species can individually be recognized based on image data obtained independently of solvent conditions and the complexities of EPR spectra. These data not only present deductive knowledge for individual spectra of free-radical intermediates but also show that a proper irradiation frequency can be selected so as to selectively image a species of interest and avoid the redundancy of a species of no interest. This method is significantly superior to ¹H MRSI that needs to employ special pulse sequences so as to suppress water proton signals so that weak signals from metabolic products such as choline, lactates and citrates can be collected.

In order to further test the ReMI capability of imaging free-radicals, phantoms shown in item e in FIG. 2 were used, wherein ReMI images were obtained by continuously irradiating EPR at the intervals of 1 MHz in the range from 527.5 MHz to 537.5 MHz (items f-h in FIG. 2). In each image obtained in this frequency range, it is clear that the intensity declined as the EPR absorption of each free-radical decreased. This result demonstrates that ReMI can characterize individual free-radical intermediates by sweeping EPR irradiation frequencies.

### 3. Metabolic Imaging Using Mitochondria

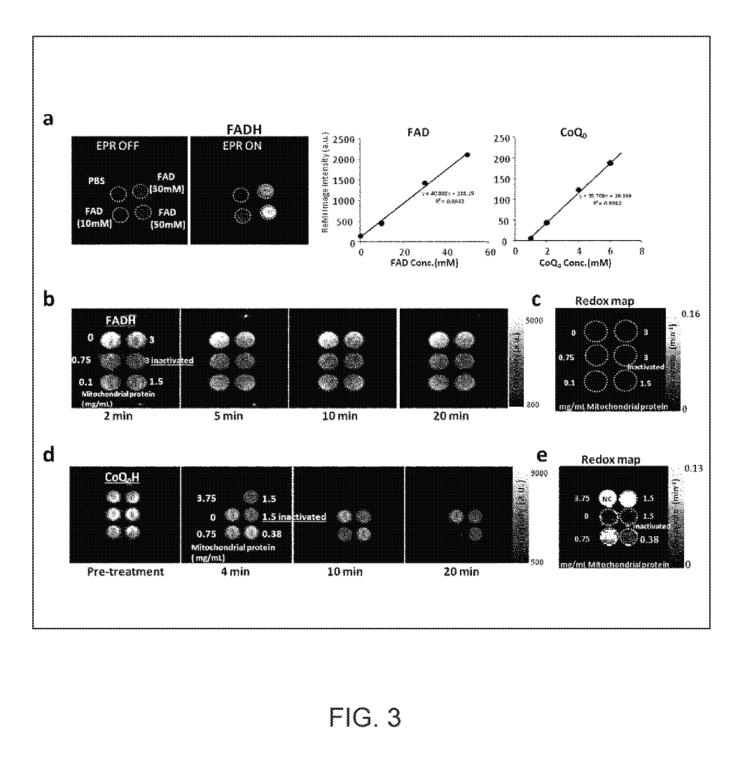

In order to monitor real-time oxidation/reduction reactions, reactions of free-radical intermediates FADH and CoQ₀H with mitochondria were examined. Phantoms were composed of six tubes disposed in two columns, and those tubes were composed of mitochondria fractions having various concentrations that were reacted with FADH or CoQ₀H. Item a in FIG. 3 shows that ReMI image intensity increases dependently on free-radical concentrations. ReMI images were obtained over time after starting the reaction of mitochondria with FADH or CoQ₀H. When FAD free-radicals were added, there was no reaction with mitochondria, while when CoQ₀H was used, the image intensity declined dependently on the concentration of mitochondria (item d in FIG. 3 and item e in FIG. 3). When an inactivated mitochondria fraction was used, there was no decline in the image intensity just like a control (middle row, right column). As used herein, the “redox map” refers to the intensity declining speed and showed no change in FADH, while when CoQ₀H was used, it increased dependently of the concentration of mitochondria (item c in FIG. 3 and item e in FIG. 3). Thus, the images of metabolic rates by ReMI totally differ between FADH and CoQ₀H, and the conversion of CoQ₀H to CoQ₀H₂ in mitochondria can be visualized with an ReMI map (item d in FIG. 3 and item e in FIG. 3).

### Metabolic Imaging in Mice Using Sequential ReMI

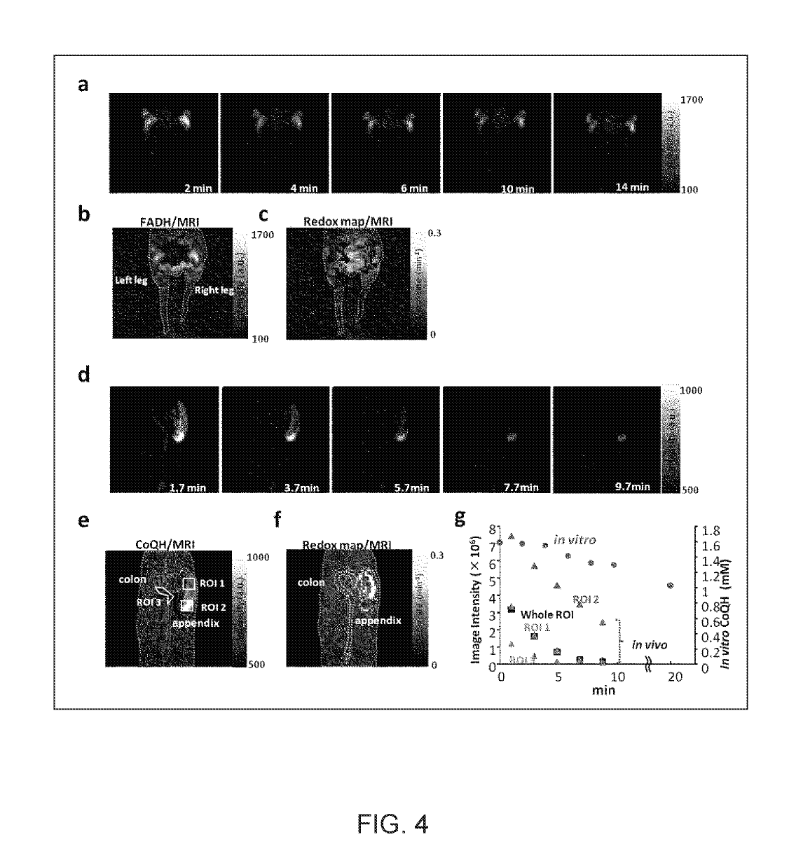

FADH and CoQ₀H were administered to mice and then ReMI imaging was performed every two minutes. Item a in FIG. 4 shows images taken every two minutes after FADH was intramuscularly administered in both legs. Image intensities from those two places were stable during the test period (14 minutes). Item b in FIG. 4 shows anatomical images of FADH intensity and the oxidation/reduction rate of this intensity, which is shown as a redox map. The ReMI scanning shows that FADH is metabolized slowly in the muscle.

A similar experiment using ReMI was conducted by introducing CoQ₀H into the rectum (item d in FIG. 4). Just like its reaction with mitochondria in the phantom experiment, the intensity of CoQ₀H declined over time (item d in FIG. 3 and item e in FIG. 3). Item d in FIG. 4 shows the anatomical and metabolic ReMI images of CoQ₀H in the intestinal canal. This result coincided with the phantom experiment. The fusion images of MRI and ReMI show that FADH and CoQ₀H are site-specifically distributed in the legs and the intestine, respectively (item b in FIG. 4 and item e in FIG. 4).

The pharmacokinetic characteristics of FADH and CoQ₀H were determined by their decreasing rates and were different from each other. The pharmacokinetic map of CoQ₀H was significantly dependent on tissue sites, while the pharmacokinetic map of FADH was constant (item c in FIG. 4 and item fin FIG. 4). Free-radical intermediates might lose their paramagnetism as a result of electron transfer and/or oxidation/reduction reaction, redistribution and discharge in mitochondria, and some of them might possibly induce a sudden decline in CoQ₀H in the mouse intestine. As shown in item g in FIG. 4, the sequential plot of changes in the image intensity of ReMI was different in the living body among the whole region of interest (ROI), the upper region of the appendix (ROI-1), the lower region of the appendix (ROI-2) and the colon (ROI-3), while the CoQ₀H solution was stable (item e in FIG. 3).

### 5. Visualization of Vitamin K₁ by ReMI

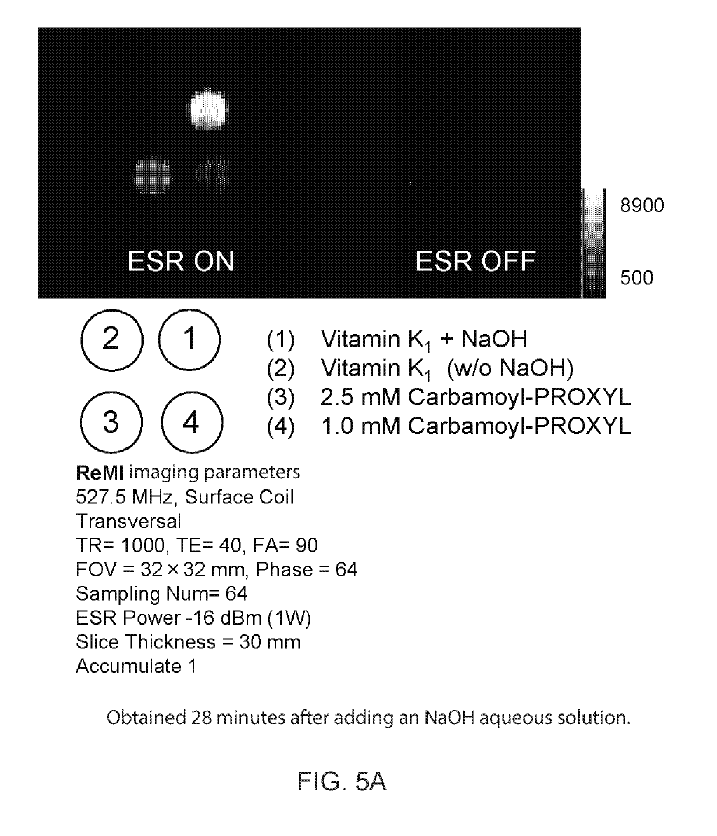

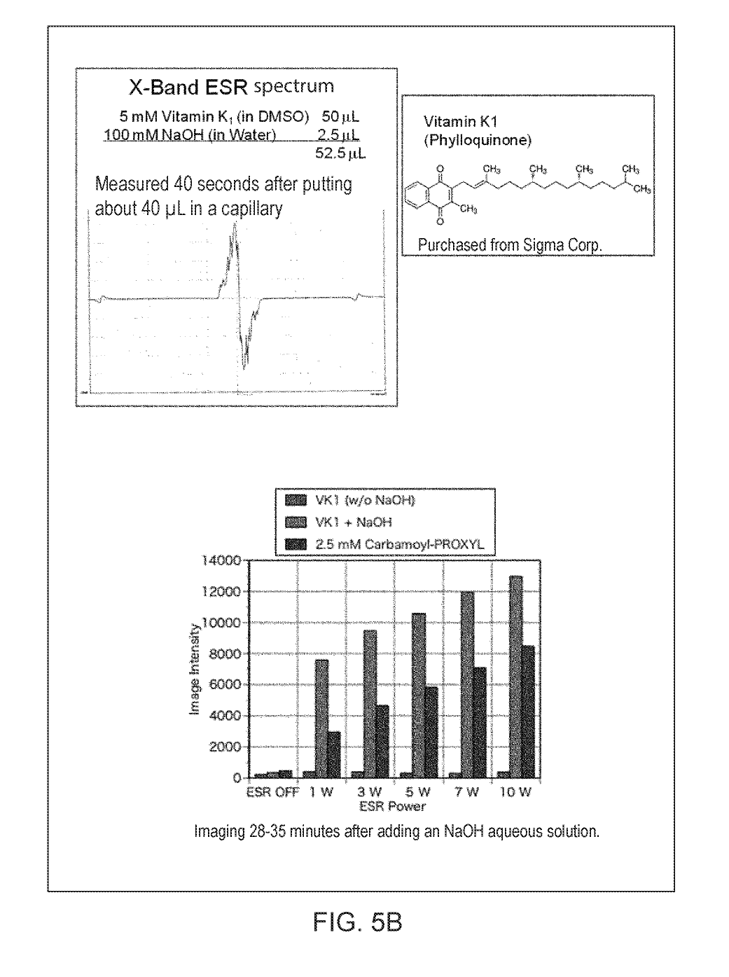

Next, the present inventors visualized vitamin K₁ using ReMI. FIG. 5A shows the result. The image at left is an image taken when ESR irradiation was ON, and an image at right is an image taken when ESR irradiation was OFF. In this working example, vitamin K₁ was dissolved in DMSO, which is an organic solvent, and a NaOH solution was added as a redox material. The following shows the composition. The final concentrations of vitamin K₁ and NaOH were 4.76 mM.

| | | |
|---|---|---|
| 5 mM | Vitamin K₁ (in DMSO) | 500 μL |
| 100 mM | NaOH (in water) | 25 μL |
| | | 525 μL |

Immediately after mixing, 500 μL was placed in a Durham tube, sealed and placed in a resonator, and then ReMI imaging was performed. FIG. 5A is an image obtained 28 minutes after adding a NaOH solution.

The present working example shows that a vitamin K₁ radical can be observed well when NaOH is added as a redox material in an organic solvent, i.e., in a lipid environment.

FIG. 5B shows an X-band ESR spectrum as well as image intensities when the ESR irradiation intensity was changed in the present working example.

### 6. Visualization of Vitamin K₂ by ReMI

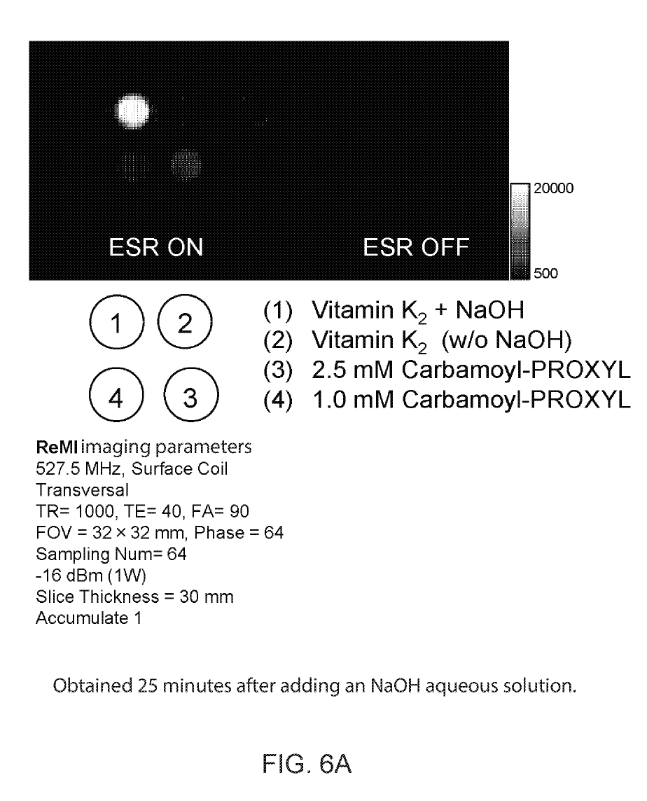

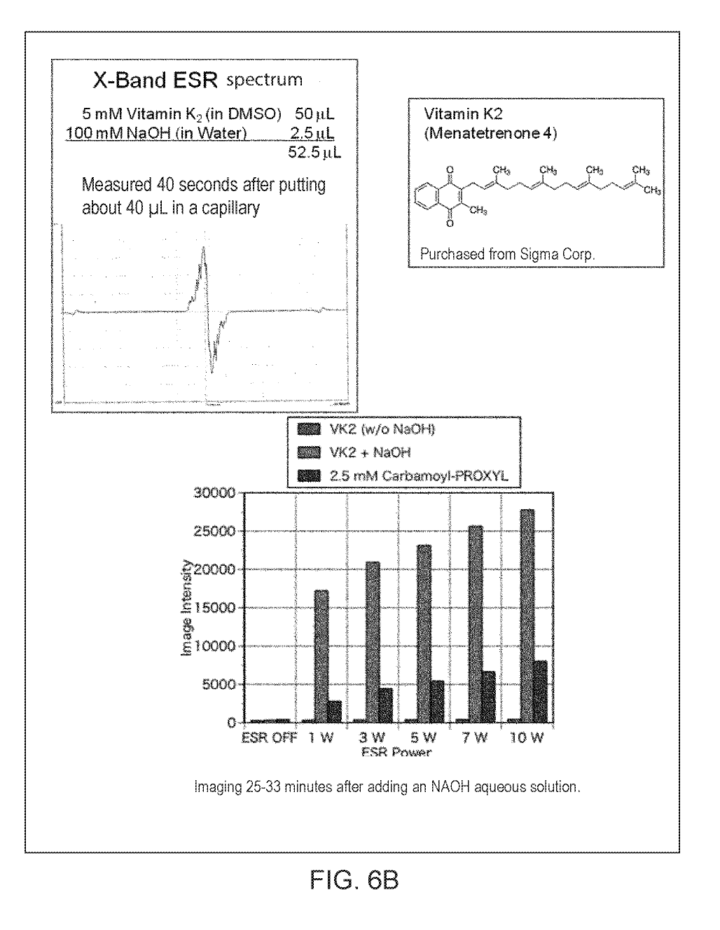

Next, the present inventors visualized vitamin K₂ using ReMI. FIG. 6A shows the result. The image at left is an image taken when ESR irradiation was ON, and an image at right is an image taken when ESR irradiation was OFF. In this working example, vitamin K₂ powder was dissolved in DMSO, which is an organic solvent, and a NaOH solution was added as a redox material. The following shows the composition. The final concentrations of vitamin K₂ and NaOH were 4.76 mM.

| | | |
|---|---|---|
| 5 mM | Vitamin K₂ (in DMSO) | 500 μL |
| 100 mM | NaOH (in water) | 25 μL |
| | | 525 μL |

Immediately after mixing, 500 μL, was placed in a Durham tube, sealed and placed in a resonator, and then ReMI imaging was performed. FIG. 6A is an image obtained 25 minutes after adding a NaOH solution.

The present working example shows that a vitamin K₂ radical can be observed well when NaOH is added as a redox material in an organic solvent, i.e., in a lipid environment.

FIG. 6B shows an X-band ESR spectrum as well as image intensities when the ESR irradiation intensity was changed in the present working example.

### 7. Visualization of Vitamin K₃ by ReMI

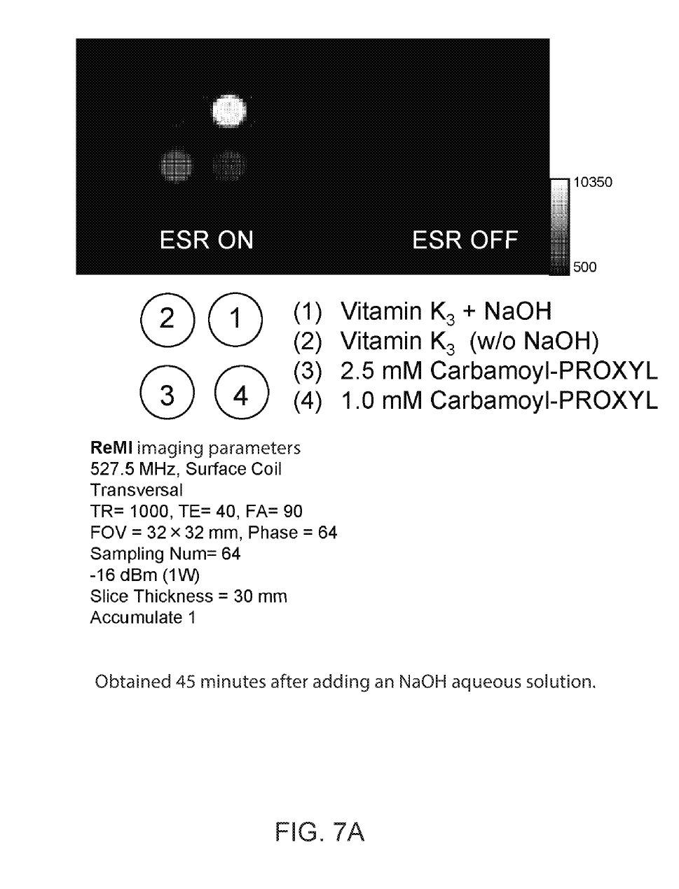

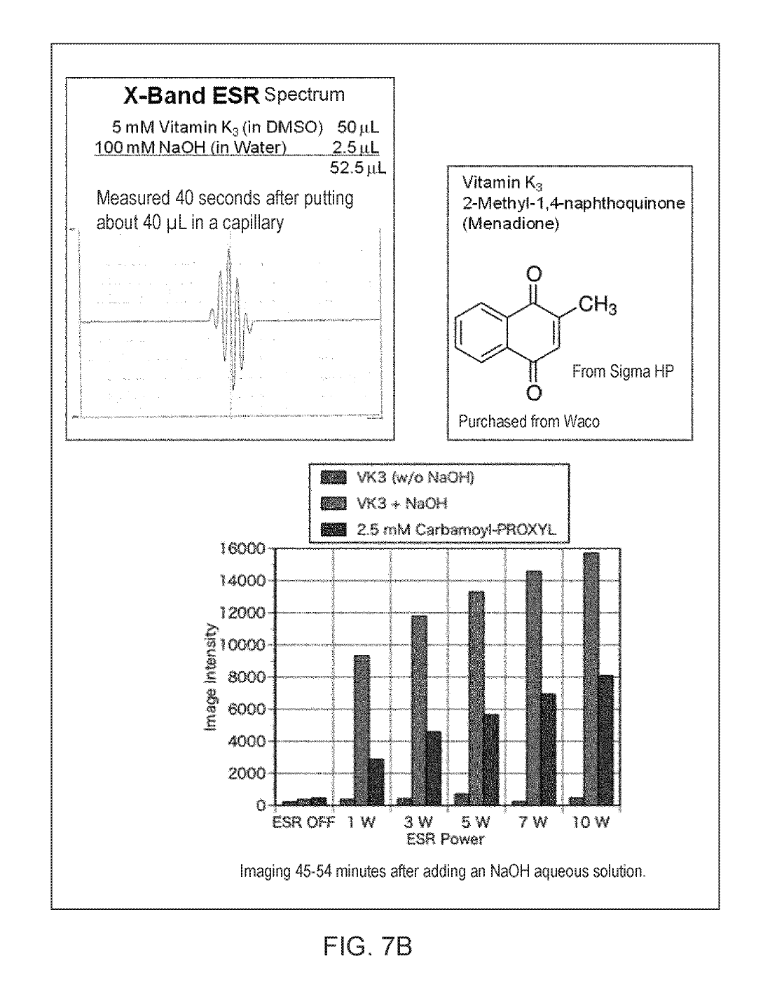

Next, the present inventors visualized vitamin K₃ using ReMI. FIG. 7A shows the result. The image at left is an image taken when ESR irradiation was ON, and an image at right is an image taken when ESR irradiation was OFF. In this working example, vitamin K₃ was dissolved in DMSO, which is an organic solvent, and an NaOH solution was added as a redox material. The following shows the composition. The final concentrations of vitamin K₃ and NaOH were 4.76 mM.

| | | |
|---|---|---|
| 5 mM | Vitamin K₃ (in DMSO) | 500 μL |
| 100 mM | NaOH (in water) | 25 μL |
| | | 525 μL |

Immediately after mixing, 500 μL, was placed in a Durham tube, sealed andset placed in a resonator, and then ReMI imaging was performed. FIG. 7A is an image obtained 45 minutes after adding a NaOH solution.

The present working example shows that a vitamin K₃ radical can be observed well when NaOH is added as a redox material in an organic solvent, i.e., in a lipid environment.

FIG. 7B shows an X-band ESR spectrum as well as image intensities when the ESR irradiation intensity was changed in the present working example.

### 8. Visualization of Vitamin K₃ by ReMI

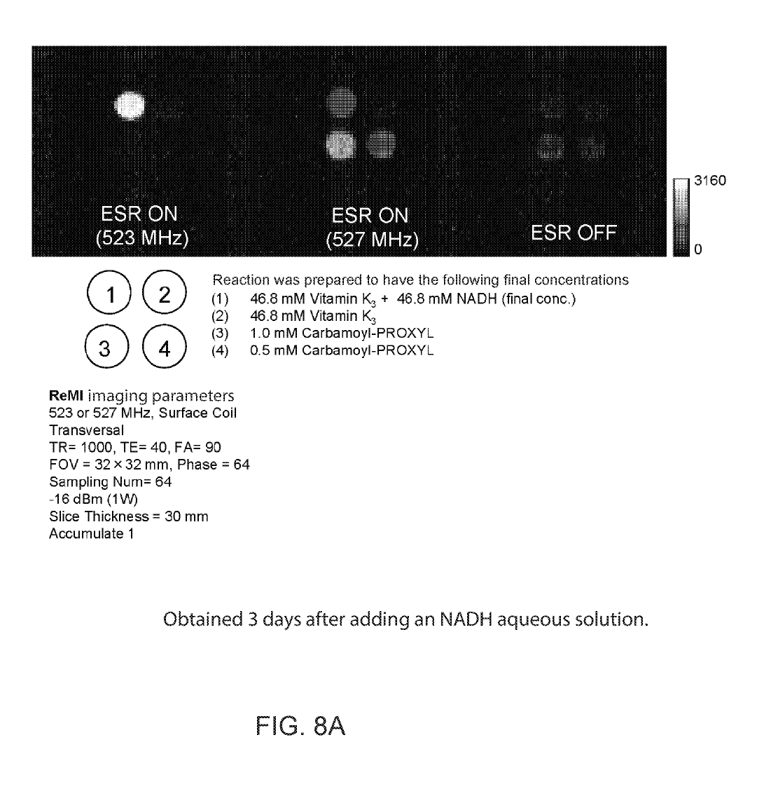

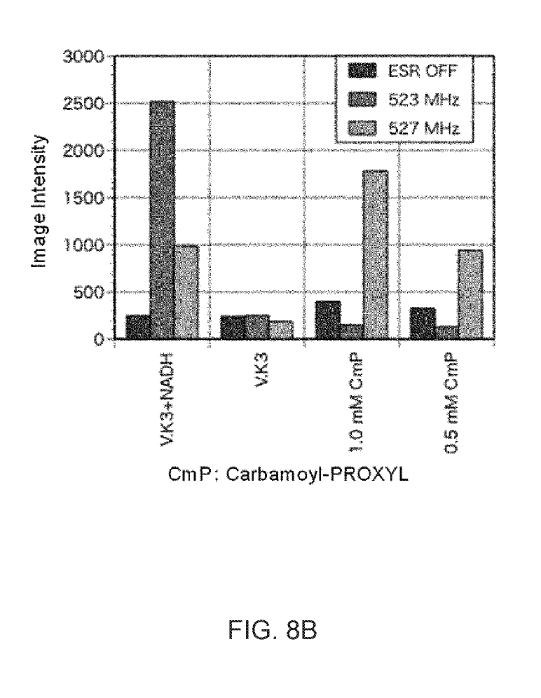

Furthermore, the present inventors changed the frequency of ESR irradiation and visualized vitamin K₃ using ReMI. FIG. 8A shows the result. The image at left is an image taken when ESR irradiation was ON (523 MHz), an image in the middle is an image taken when ESR irradiation was ON (527 MHz), and an image at right is an image taken when ESR irradiation was OFF. In this working example, vitamin K₃ was dissolved in DMSO, which is an organic solvent, and a NaOH solution was added as a redox material. The following shows the composition. The final concentrations of vitamin K₃ and NaOH were 46.8 mM.

| | | |
|---|---|---|
| 50 mM | Vitamin K₃ (in DMSO) | 504 μL |
| 720 mM | NaOH (in water) | 35 μL |
| | | 539 μL |

Immediately after mixing, 500 μL, was placed in a Durham tube, sealed and placed in a resonator, and then ReMI imaging was performed. FIG. 8A is an image obtained 3 days after adding a NaOH solution.

The present working example shows that a free-radical intermediate of interest can selectively be imaged by adjusting the frequency of ESR irradiation. FIG. 8B is a graph showing image intensities in the present working example.

9. Visualization of Vitamin K₂ and Vitamin K₃ by ReMI

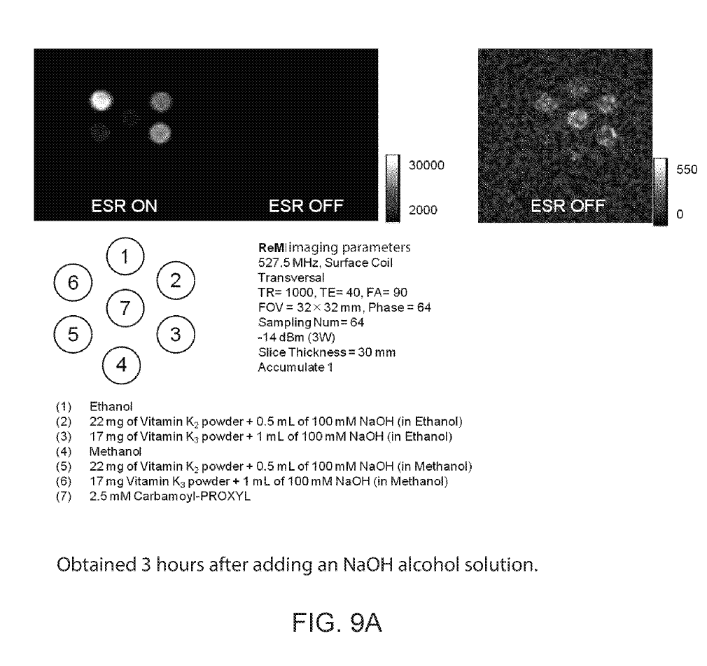

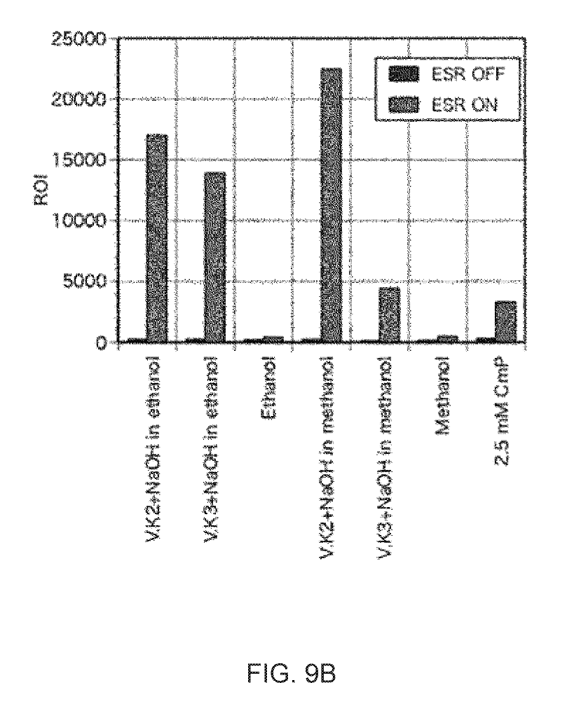

Next, the present inventors visualized vitamin K₂ and vitamin K₃ at the same time using ReMI. FIG. 9A shows the result. The image at left is an image taken when ESR irradiation was ON, and an image at right is an image taken when ESR irradiation was OFF. In this working example, vitamin K₂ or vitamin K₃ powder was added to an NaOH alcohol solution prepared by dissolving NaOH in ethanol or methanol, which is an organic solvent. The reaction solution was adjusted such that the final concentrations of vitamin K₂ and vitamin K₃ became 100 mM.

Immediately after mixing, 300 μL was placed in a Durham tube, sealed and placed in a resonator, and then ReMI imaging was performed. FIG. 9A is an image obtained 3 hours after adding an NaOH alcohol solution.

The present working example shows that a plurality of free-radical intermediates can be observed well by ReMI in a lipid environment. FIG. 9B is a graph showing image intensities in the present working example. In each column, the left is when ESR irradiation was OFF, and the right is when ESR irradiation was ON.

### 10. Visualization of Riboflavin (Vitamin B₂) by ReMI

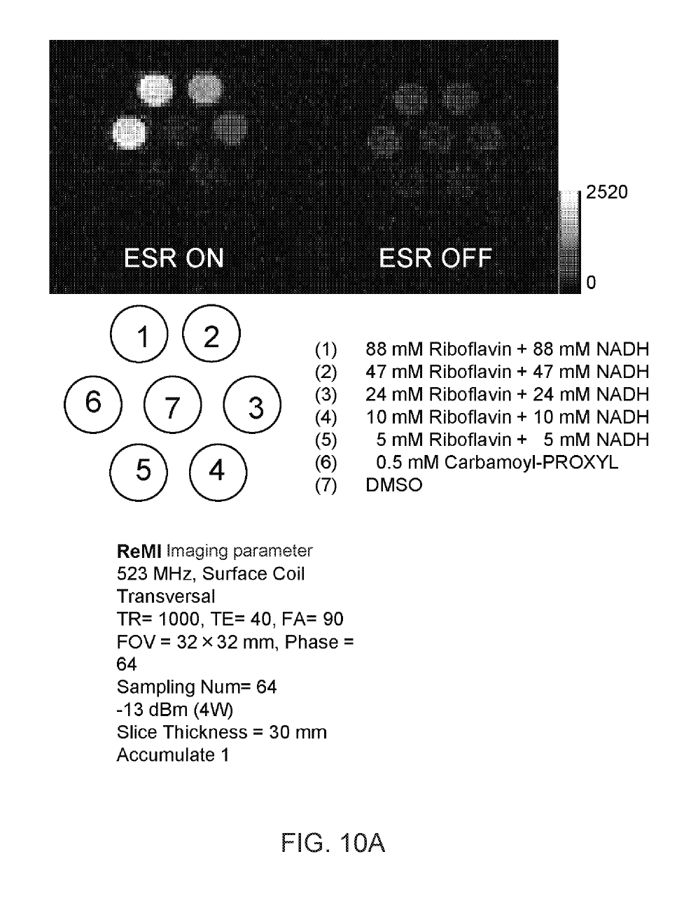

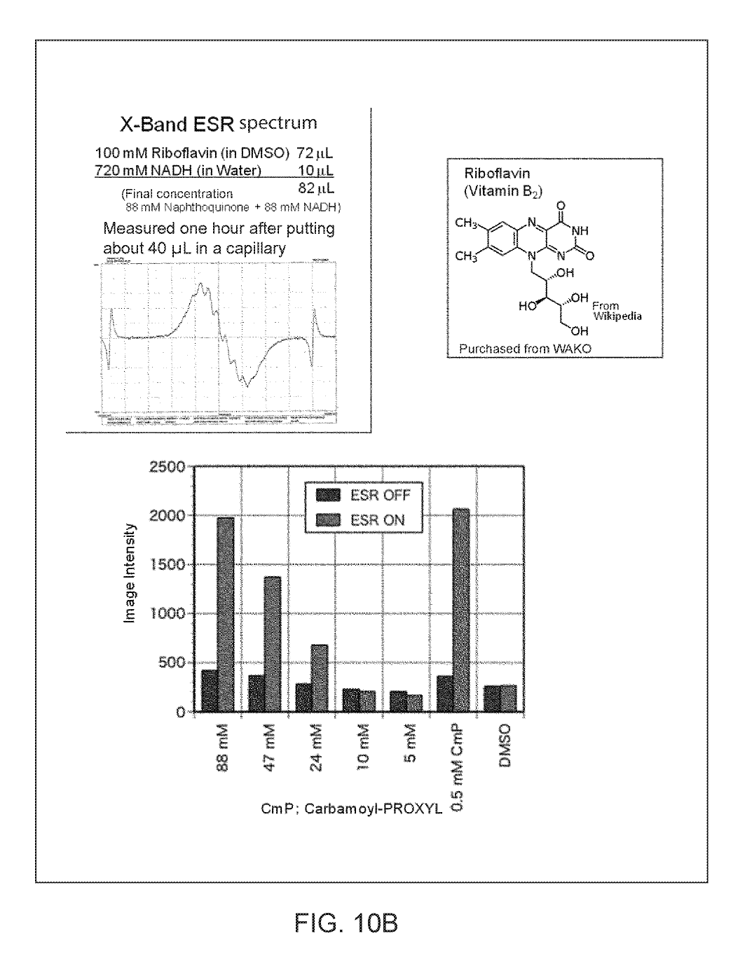

Next, the present inventors visualized a riboflavin (vitamin B₂) radical using ReMI. FIG. 10A shows the result. The image at left is an image taken when ESR irradiation was ON, and an image at right is an image taken when ESR irradiation was OFF. In this working example, riboflavin powder was dissolved in DMSO, which is an organic solvent, and an NADH aqueous solution was added as a redox material.

Immediately after mixing, 300 μL was placed in a Durham tube, sealed and placed in a resonator, and then ReMI imaging was performed. FIG. 10A is an image obtained 3 hours after adding an NaOH solution.

The present working example shows that a riboflavin (vitamin B₂) radical can be observed well when NADH is added as a redox material in an organic solvent, i.e., in a lipid environment.

FIG. 10B shows an X-band ESR spectrum and a graph showing image intensities in the present working example. In the graph of image intensities, the left is when ESR irradiation was OFF and the right when ESR irradiation was ON in each column.

### 11. Visualization of Epigallocatechin Gallate (EGCG) by ReMI

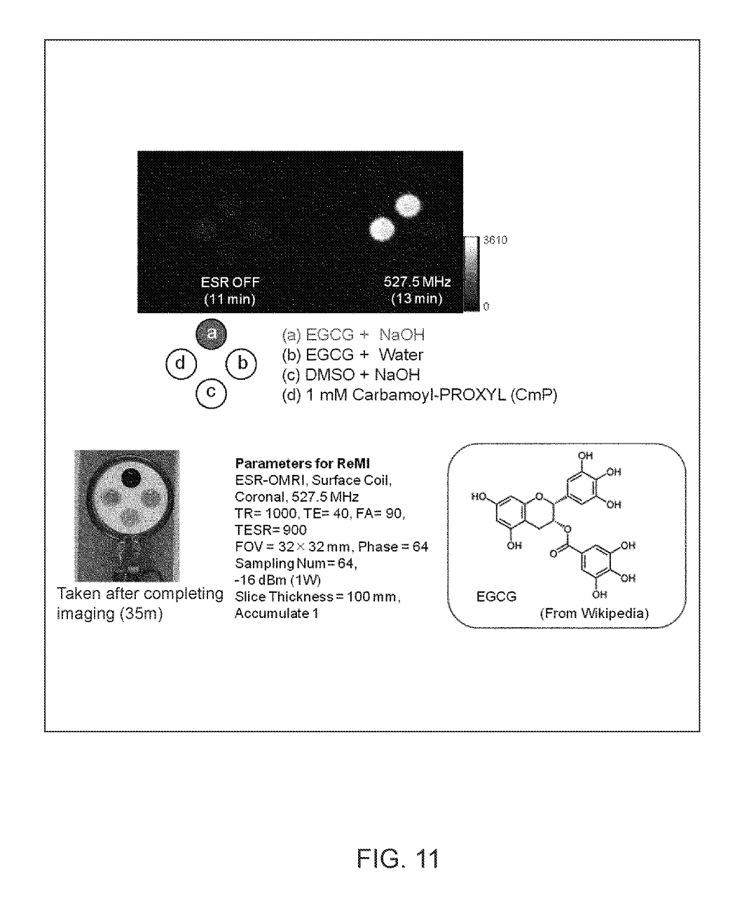

Next, the present inventors visualized epigallocatechin gallate using ReMI. FIG. 11 shows the result. The image at left is an image taken when ESR irradiation was OFF, and an image at right is an image taken when ESR irradiation was ON. In this working example, epigallocatechin gallate was dissolved in DMSO, which is an organic solvent, and an NaOH solution was added as a redox material. The following shows the composition.

| | | |
|---|---|---|
| 25 mM | EGCG (in DMSO) | 270 μL |
| 1M | NaOH (in water) | 30 μL |
| | | 300 μL |

The present working example shows that an epigallocatechin gallate radical can be observed well when NaOH is added as a redox material in an organic solvent, i.e., in a lipid environment.

### 12. Visualization of Dopamine by ReMI

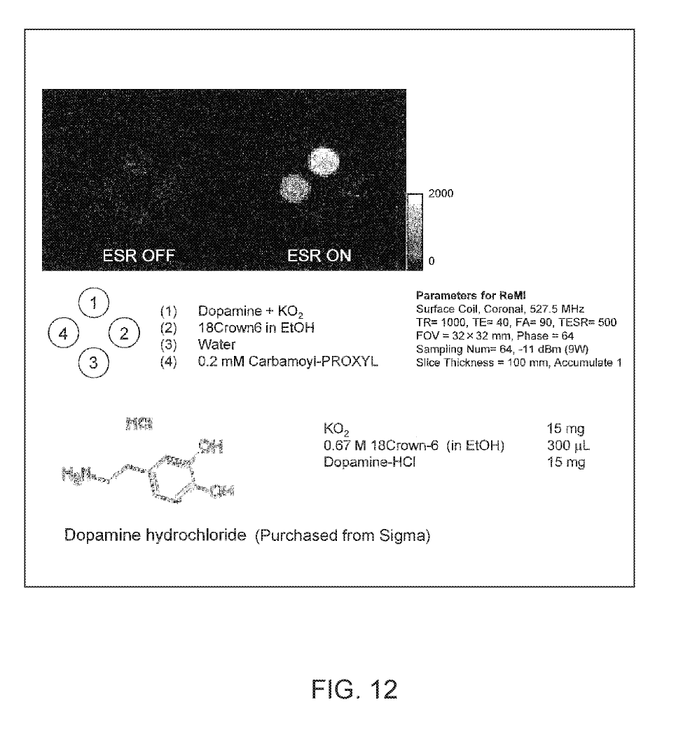

Next, the present inventors visualized dopamine using ReMI. FIG. 12 shows the result. The image at left is an image taken when ESR irradiation was OFF, and an image at right is an image taken when ESR irradiation was ON. In this working example, dopamine was dissolved in ethanol, which is an organic solvent, and a KO₂ solution was added as a redox material.

The present working example shows that a dopamine radical can be observed well when KO₂ is added as a redox material in an organic solvent, i.e., in a lipid environment.

### 13. Visualization of Chlorogenic Acid by ReMI

Next, the present inventors visualized chlorogenic acid using ReMI. FIG. 13 shows the result. The image at left is an image taken when ESR irradiation was OFF, and an image at right is an image taken when ESR irradiation was ON. In this working example, chlorogenic acid was dissolved in DMSO, which is an organic solvent, and an NaOH solution was added as a redox material. The following shows the composition.

| | | |
|---|---|---|
| 25 mM | chlorogenic acid (in DMSO) | 285 μL |
| 1M | NaOH (in water) | 15 μL |
| | | 300 μL |

The present working example shows that a chlorogenic acid radical can be observed well when NaOH is added as a redox material in an organic solvent, i.e., in a lipid environment.

### 14. Visualization of Caffeic Acid by ReMI

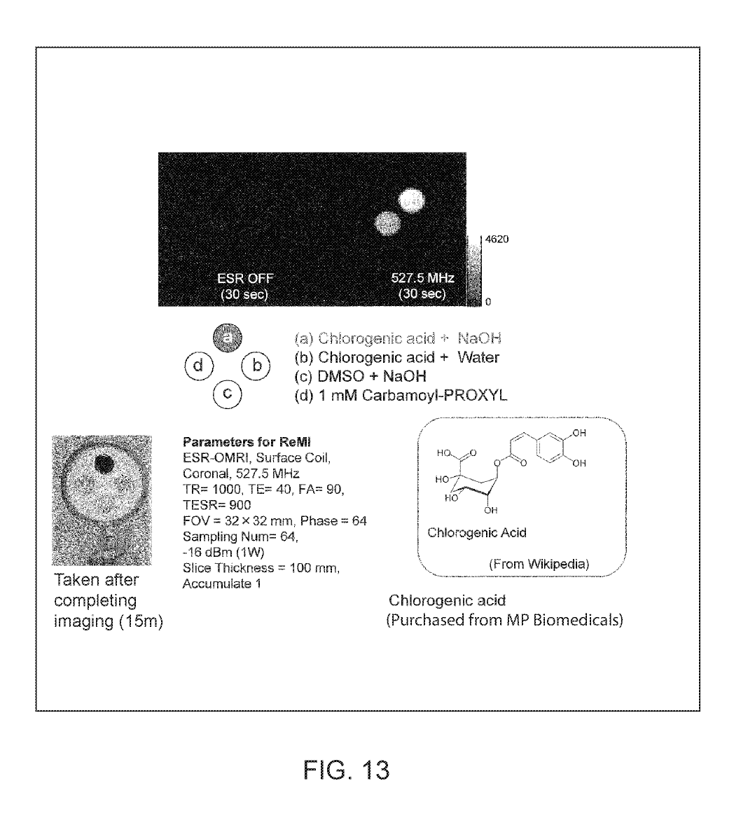

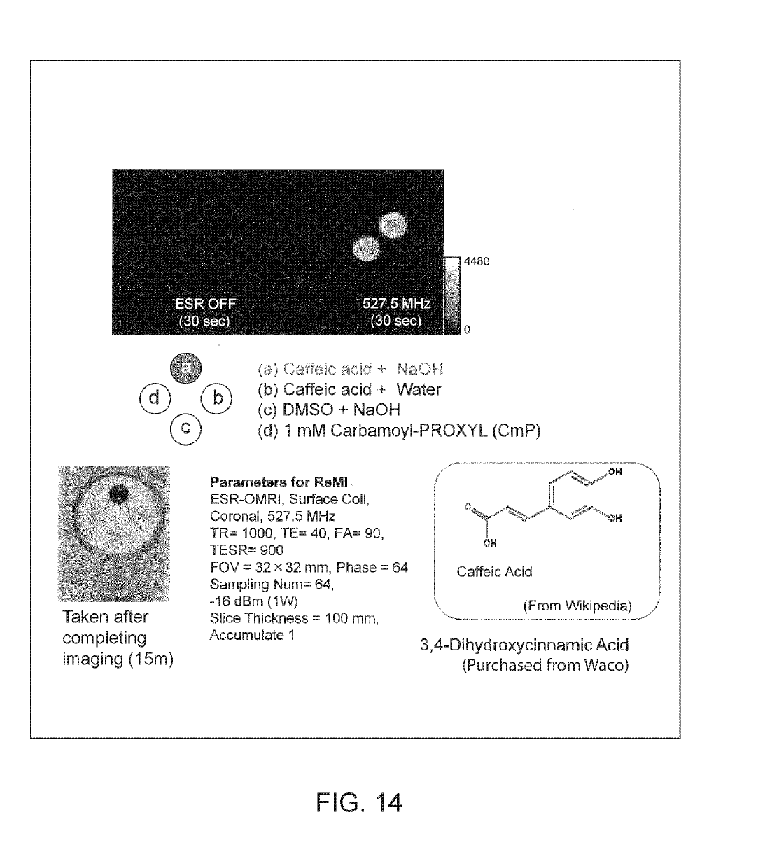

Next, the present inventors visualized caffeic acid using ReMI. FIG. 14 shows the result. The image at left is an image taken when ESR irradiation was OFF, and an image at right is an image taken when ESR irradiation was ON. In this working example, caffeic acid was dissolved in DMSO, which is an organic solvent, and an NaOH solution was added as a redox material. The following shows the composition.

| | | |
|---|---|---|
| 25 mM | caffeic acid (in DMSO) | 285 μL |
| 1M | NaOH (in water) | 15 μL |
| | | 300 μL |

The present working example shows that a caffeic acid radical can be observed well when NaOH is added as a redox material in an organic solvent, i.e., in a lipid environment.

### 15. Visualization of Rosmarinic Acid by ReMI

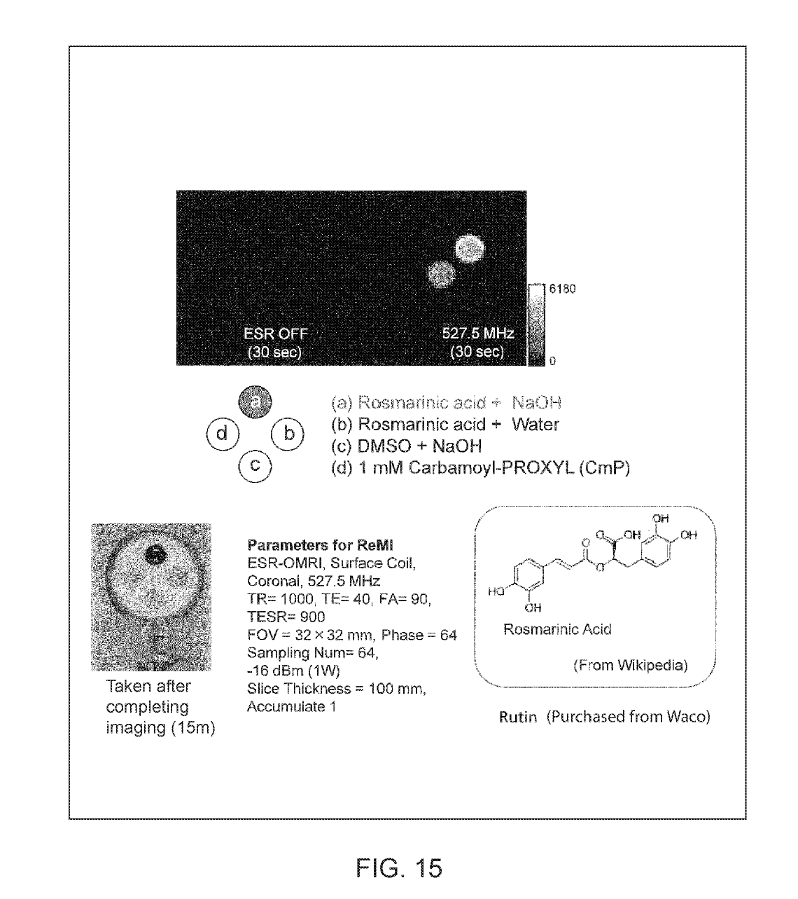

Next, the present inventors visualized rosmarinic acid using ReMI. FIG. 15 shows the result. The image at left is an image taken when ESR irradiation was OFF, and an image at right is an image taken when ESR irradiation was ON. In this working example, rosmarinic acid was dissolved in DMSO, which is an organic solvent, and an NaOH solution was added as a redox material. The following shows the composition.

| | | |
|---|---|---|
| 25 mM | rosmarinic acid (in DMSO) | 277.5 μL |
| 1M | NaOH (in water) | 22.5 μL |
| | | 300 μL |

The present working example shows that a rosmarinic acid radical can be observed well when NaOH is added as a redox material in an organic solvent, i.e., in a lipid environment.

### 16. Visualization of Rutin by ReMI

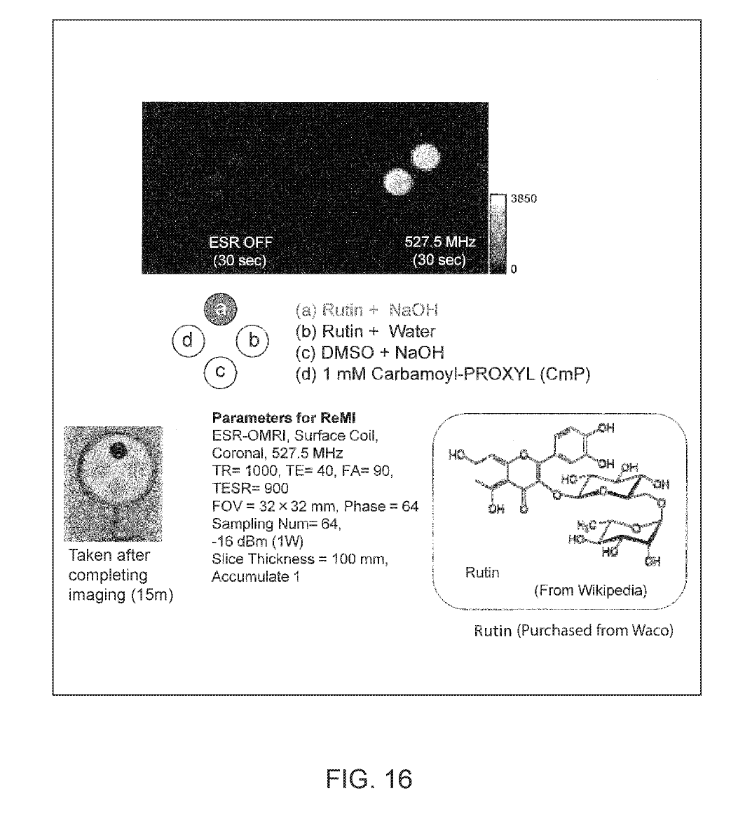

Next, the present inventors visualized rutin using ReMI. FIG. 16 shows the result. The image at left is an image taken when ESR irradiation was OFF, and an image at right is an image taken when ESR irradiation was ON. In this working example, rutin was dissolved in DMSO, which is an organic solvent, and an NaOH solution was added as a redox material. The following shows the composition.

| | | |
|---|---|---|
| 25 mM | rutin (in DMSO) | 285 μL |
| 1M | NaOH (in water) | 15 μL |
| | | 300 μL |

The present working example shows that a rutin radical can be observed well when NaOH is added as a redox material in an organic solvent, i.e., in a lipid environment.

### 17. Visualization of Seratrodast by ReMI

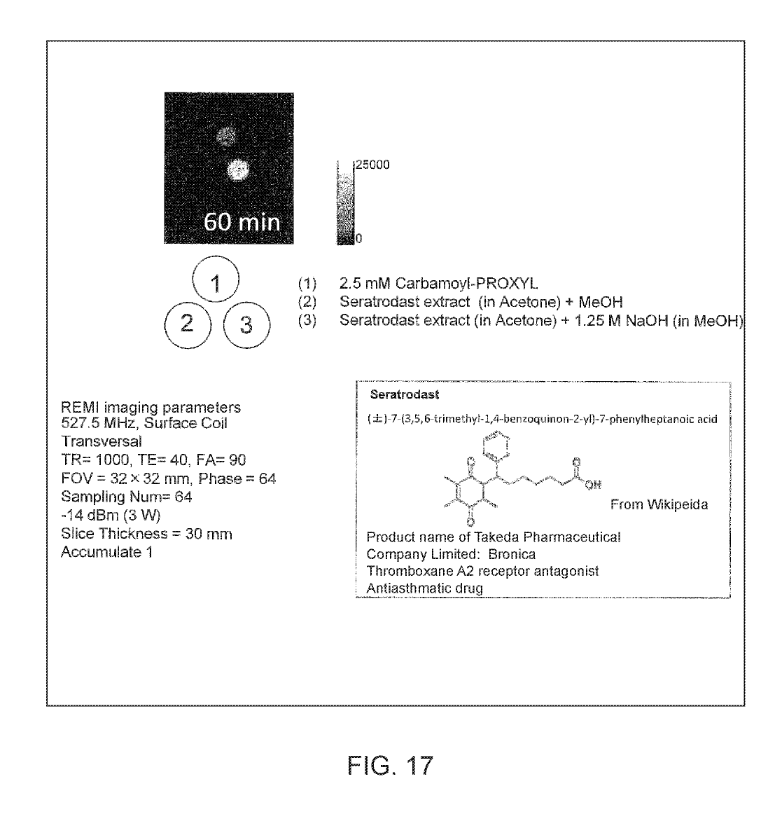

Next, the present inventors visualized seratrodast using ReMI. FIG. 17 shows the result. In this working example, seratrodast was dissolved in acetone, which is an organic solvent, and an NaOH solution was added as a redox material.

The present working example shows that a seratrodast radical can be observed well when NaOH is added as a redox material in an organic solvent, i.e., in a lipid environment.

### 18. Visualization of Trolox by ReMI

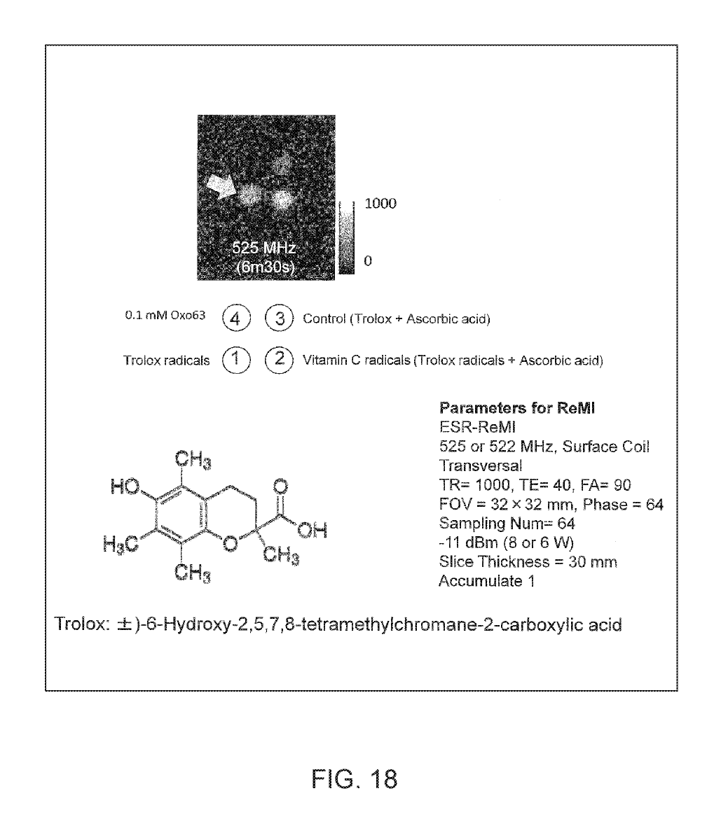

Next, the present inventors visualized trolox using ReMI. FIG. 18 shows the result. In this working example, trolox was dissolved in 18-crown-6/ethanol, which is an organic solvent, and KO₂ was added as a redox material.

The present working example shows that a trolox radical can be observed well when KO₂ is added as a redox material in an organic solvent, i.e., in a lipid environment.

19. ReMI Image when TEMPOL is Dissolved in an Organic Solvent

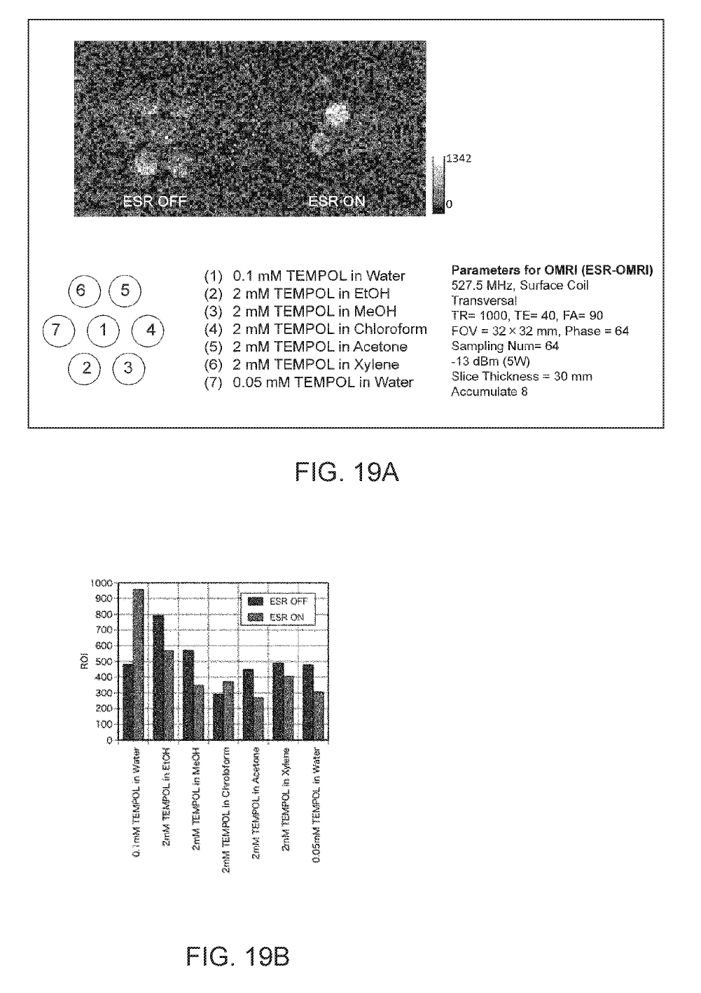

Next, as a comparative example, the present inventors dissolved TEMPOL, which is a nitroxyl radical, in an organic solvent and performed ReMI imaging. FIG. 19A shows the result. The image at left is an image taken when ESR irradiation was OFF, and an image at right is an image taken when ESR irradiation was ON. In this working example, various concentrations of TEMPOL were dissolved in various organic solvents (ethanol, methanol, chloroform, acetone and xylene) and water as a control.

The present working example shows that the image intensity of TEMPOL dramatically declined in any organic solvent as compared with a case in which it was dissolved in water. FIG. 19B is a graph showing image intensities in the present working example. In each column, the left is when ESR irradiation was OFF, and the right is when ESR irradiation was ON.

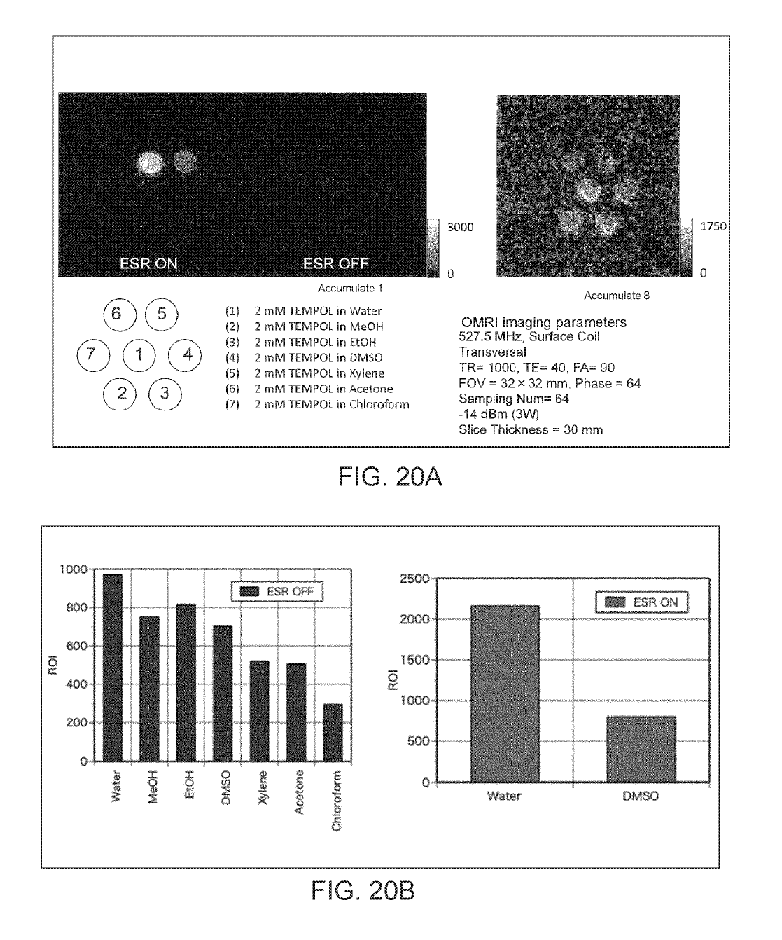

Next, DMSO was used as a redox material, and TEMPOL was dissolved in various organic solvents in a similar manner, and then ReMI imaging was performed. FIG. 20A shows the result. The image at left is an image taken when ESR irradiation was ON, and an image at right is an image taken when ESR irradiation was OFF.

The present working example shows that the image intensity of TEMPOL declined to about ⅓ even when TEMPOL was dissolved in DMSO as compared with a case in which it was dissolved in water. FIG. 20B is graphs showing image intensities in the present working example. The left graph is when ESR irradiation was OFF, and the right graph is when ESR irradiation was ON.

18. ReMI Image when MC-PROXYL is Dissolved in an Organic Solvent

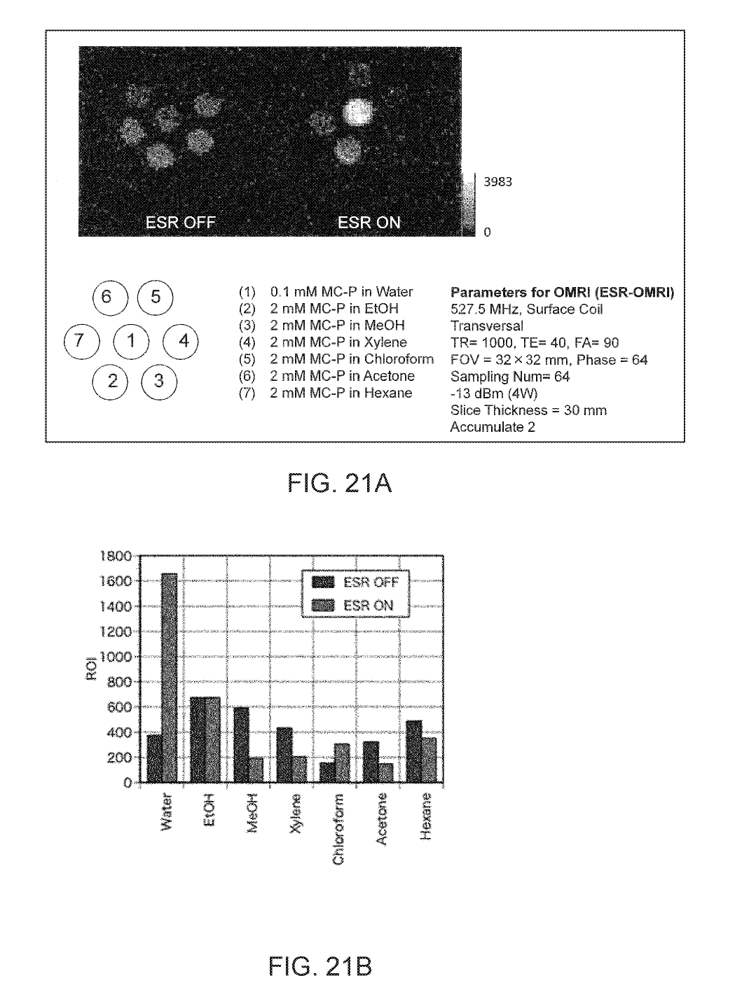

Next, as a comparative example, the present inventors dissolved MC-PROXYL, which is a nitroxyl radical, in an organic solvent and performed ReMI imaging. FIG. 21A shows the result. The image at left is an image taken when ESR irradiation was OFF, and an image at right is an image taken when ESR irradiation was ON. In this working example, various concentrations of MC-PROXYL were dissolved in various organic solvents (ethanol, methanol, chloroform, acetone, xylene and hexane) and water as a control.

The present working example shows that the image intensity of MC-PROXYL dramatically declined in any organic solvent as compared with a case in which it was dissolved in water. FIG. 21B is a graph showing image intensities in the present working example. In each column, the left is when ESR irradiation was OFF, and the right is when ESR irradiation was ON.

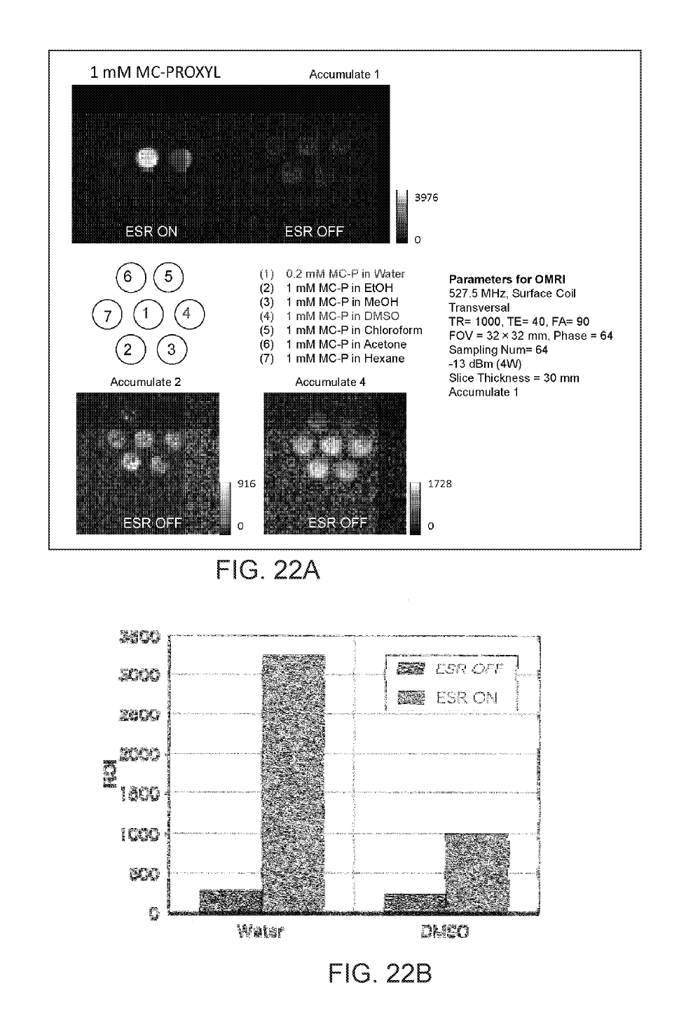

Next, DMSO was used as a redox material, and MC-PROXYL was dissolved in various organic solvents in a similar manner, and then ReMI imaging was performed. FIG. 22A shows the result. The image at left is an image taken when ESR irradiation was ON, and an image at right is an image taken when ESR irradiation was OFF.

The present working example shows that the image intensity of MC-PROXYL dramatically declined even when MC-PROXYL was dissolved in DMSO as compared with a case in which it was dissolved in water. FIG. 22B is a graph showing image intensities in the present working example. In each column, the left is when ESR irradiation was OFF, and the right is when ESR irradiation was ON.

It goes without saying that the present invention can be modified in various manners without being limited by the abovementioned embodiment as far as those modifications do not depart from the scope of the present invention.

What is claimed is:

1. A method for detecting an oxidation/reduction reaction of a molecule that undergoes a free-radical reaction in a lipid environment, the method comprising:
obtaining a proton image of the molecule that undergoes a free-radical reaction in a lipid environment by applying a magnetic resonance method to a living body or sample to be examined; and
examining the image intensity of the living body or sample in the proton image.

2. The method according to claim 1, wherein the step of obtaining a proton image is to obtain two or more proton images over time, and the method further comprises comparing sequential changes in the image intensity of the living body or sample in the proton images.

3. The method according to claim 1, wherein the magnetic resonance method is Overhauser MRI, and the step of obtaining a proton image is to obtain a proton image in which an electro spin of the molecule that undergoes a free-radical reaction in a lipid environment is excited.

4. The method according to claim 3 further comprising:
obtaining a proton image in which an electron spin of the molecule that undergoes a free-radical reaction in a lipid environment is not excited; and
comparing between the proton image in which an electron spin of the molecule that undergoes a free-radical reaction in a lipid environment is excited and the proton image in which an electron spin of the molecule that undergoes a free-radical reaction in a lipid environment is not excited and then calculating a difference or percentage of the image intensity of the living body or sample in the two images.

5. The method according to claim 1, wherein the molecule that undergoes a free-radical reaction in a lipid environment is a molecule having a quinone skeleton.

6. The method according to claim 5, wherein the molecule having a quinone skeleton is selected from the group consisting of ubiquinone (CoQ₁₀), riboflavin, vitamin K₁, vitamin K₂, vitamin K₃, 1,4-benzoquinone (p-quinone), 2,6-dichloro-p-quinone, 1,4-naphthoquinone and seratrodast.

7. The method according to claim 1, wherein the step of obtaining a proton image is to obtain proton images of two or more molecules that undergo a radical reaction in a lipid environment.

8. The method according to claim 1, further comprising obtaining a proton image of a molecule that undergoes a radical reaction in an aquatic environment.

9. The method according to claim 1, wherein the living body or sample is administered with a redox material in advance.

10. The method according to claim 9, wherein the living body or sample is administered with the molecule that undergoes a free-radical reaction in a lipid environment in advance.

11. The method according to claim 9, wherein the redox material is selected from the group consisting of NaOH, NADH, KO₂ and combinations thereof.

12. The method according to claim 1, wherein the molecule that undergoes a free-radical reaction in a lipid environment is dissolved in a solvent selected from the group consisting of ethanol, methanol, DMSO, acetone, hexane, chloroform, alkaline solutions and combinations thereof.
